(19) United States
(12) Patent Application Publication
(10) Pub. No.: US 2015/0133341 A1
(43) Pub. Date: May 14, 2015

(54) Intra-Operative Cancer Diagnosis Based on a Hyperpolarized Marker

(71) Applicant: ALBEDA INNOVATIONS APS, Copenhagen (DK)

(72) Inventors: Mathilde Lerche, Frederiksberg C (DK); Magnus Karlsson, Malmoe (SE); Pernille Rose Jensen, Vaerloese (DK)

(21) Appl. No.: 14/399,240

(22) PCT Filed: May 7, 2013
(86) PCT No.: PCT/EP2013/059481

§ 371 Date: Nov. 6, 2014

(30) Foreign Application Priority Data
May 7, 2012 (EP) 12166936.0
Jan. 31, 2013 (EP) 13153457.0

(51) Int. Cl. G01N 33/84; G01N 33/50

(52) U.S. Cl. CPC G01N 33/84; G01N 33/5005; G01N 2800/7028
USPC 506/10

(57) ABSTRACT

The present invention is concerned with an in vitro method of diagnosing cancer in a tissue sample, wherein said tissue sample is obtained from a patient undergoing cancer surgery. The method described herein is based on a hyperpolarized marker, which is contacted with the tissue sample, and an NMR spectrum and/or an MR image obtained of the tissue sample after having been contacted with the hyperpolarized marker.

### FIELD OF THE INVENTION

The present invention relates to a method of diagnosing cancer in a tissue sample obtained from a patient undergoing cancer surgery. The present method is carried out in vitro and can be used to provide a reliable diagnosis within a short time frame, i.e. while the operation is still ongoing. The present method relies on contacting a hyperpolarized marker with the tissue sample, and an NMR spectrum and/or an MR image obtained of the tissue sample after having been contacted with the hyperpolarized marker. By comparing the NMR spectrum and/or the MR image and/or at least one parameter determined from said spectrum and/or image to a reference, cancer can be diagnosed in the tissue sample. The invention further relates to a combination of (i) a metabolic marker indicating metabolically active cells and (ii) a metabolic marker allowing a distinction between lymphocytes and cancer cells as well as diagnostic compositions thereof.

### BACKGROUND OF THE INVENTION

A surgery is used in the majority of all cancers with different purposes, ranging from diagnosis to treatment of the cancer or to relieving the symptoms. In patients diagnosed with a specific type of cancer, particularly breast, prostate and colon cancer, excision of the primary tumor by surgery corresponds to a main method of treatment.

Clearly, it is crucial in this method of treatment that all malignant cells have been removed by the surgery. First of all, it is thus important to establish whether the primary tumor has been excised completely or whether a rim of malignant cells is still present. Secondly, there is the problem of invasion and metastasis. Thus, when cancer cells, e.g. breast cancer cells, are no longer confined to e.g. the breast, the cells will enter the lymphatic system and will in a first instance be found in the lymph nodes (regional). Such lymph nodes should clearly also be removed.

As is evident from the above, a diagnosis of the malignant state of other tissue than the primary tumor, e.g. of the rim or of a lymph node, while the surgery for the primary tumor is still ongoing, can be crucial for the further decision-making in the surgery as the method of treatment. Thus, if the surgeon removes some further tissue in addition to the primary tumor, and obtains information on the malignant state of this further tissue already during the ongoing surgery, the surgeon can e.g. decide to remove a further layer of cells or (a) further lymph node(s) if the tissue was diagnosed with cancer. This clearly improves the chances of success of the surgery since additional potential malignant cells are removed and there appears to be no need for a second surgery. For instance in Denmark, about 15% of all breast cancer patients, who undergo a primary surgery, are presently subjected to a second surgery. Thus, such a method would not only improve the chances of overall treatment success but would also save the patient the complications of a second surgery and thus improve the patient's quality of life, and would save costs.

With respect to the above described intra-operative diagnosis, there is e.g. the so-called sentinel lymph node dissection as surgical technique in breast cancer surgery. Using this technique, the surgeon finds the very first lymph node that filters fluid draining away from the area of the breast, which contains the primary breast cancer tumor to be removed. If cancer cells enter the lymph system, the sentinel lymph node is more likely than other lymph nodes to contain cancer cells. The sentinel lymph node is visualized with a tracer or a dye and removed, and a preliminary histological analysis of the sentinel lymph node is carried out. If cancer cells are found in the sentinel lymph node, then additional lymph nodes are removed. On the contrary, if no cancer cells are found in the sentinel lymph node, the likelihood of a spread of the cancer is low. In this case it is not necessary to dissect other lymph nodes, with the benefits of fewer complications for the patient.

However, the specificity of the above-described preliminary histological investigation (such as H&E stained frozen sections, immunohistochemistry on frozen sections, imprint cytology and molecular analysis) is rather low. Thus, e.g. for a preliminary histological investigation, a frozen section analysis is usually done by a single H&E stained frozen section. The detection of metastases in this setup is limited to macrometastases due to a limited sensitivity.

Another technique frequently applied in the intra-operational setting makes use of the Imprint cytology. The so-called Quickdiff stain allows detection of metastatic breast cancer tissue in the lymph nodes. Although the specificity of this method is very high, the sensitivity is even lower than the H&E staining of single frozen sections.

Several molecular analysis methods have been developed, inter alia the OSNACK19 assay (a one step nucleic acid amplification; Sysmex, Kobe, Japan). This assay is based on homogenization of lymph node samples followed by real-time amplification and quantization of CK19 mRNA directly from the lysate, with results available within 30 min for one sentinel lymph node and 40 min for 4 sentinel lymph nodes (see inter alia Tamaki et al., Clin Cancer Res 2009; 15:2879-2884).

All of the above methods have drawbacks, either in that they are only analyzing a minor fraction of the lymph node (e.g. about 1% for the H&E staining of frozen sections), or in that the assays correspond to destructive methods with regard to the analyzed tissue and do not allow for a reuse of the tissue for further types of diagnoses, such as e.g. a post-operative histopathology. Such a post-operative histopathology is a rather complex analysis which cannot be carried out intra-operatively, but which is nevertheless highly desirable since it provides further detailed information on the disease state and also on other possible diseases.

Summarizing the above, no reliable and non-invasive method of cancer diagnosis, which can be carried out during a surgical session, has been established to date.

Particularly in breast cancer with a 3% yearly growth in the number of women being diagnosed with breast cancer, and more than 1.2 million operations made on breast cancer patients worldwide per year, there is a strong need for a fast diagnosis method, which is capable of providing a reliable diagnosis of cancer intra-operatively (in order to determine the need for further surgical intervention) and of providing this diagnosis in a non-invasive manner (such that a further post-operative histopathology can still be carried out).

### OBJECTS AND SUMMARY OF THE INVENTION

The inventors of the present invention were able to solve the above need. Thus, they have surprisingly found a method of diagnosing cancer based on a hyperpolarized marker and NMR-detection comprising the steps as described herein, which provides a reliable cancer diagnosis in an excised tissue sample, which can be carried out while the surgery is ongoing, and which can be performed in a non-invasive manner.

In particular, the inventors of the present invention have surprisingly found a method of diagnosing a cancer metastasis in a lymph node tissue sample based on a specific combination of two hyperpolarized metabolic markers and NMR-detection comprising the steps as described herein, which provides a reliable diagnosis for a metastasis in an excised lymph node tissue sample, which can be carried out while the surgery is ongoing, and which can be performed in a non-invasive manner.

In a first object, the present invention is thus directed to a method of intra-operatively diagnosing cancer in a tissue sample, whereas the second object is directed to the use of such a method. A third object relates to the specific combination or a kit comprised of two hyperpolarized metabolic markers and a fourth object relates to diagnostic compositions thereof.

Thus, the present invention is concerned with a method of intra-operatively diagnosing cancer in a tissue sample, wherein said method is carried out on an excised tissue sample obtained from a patient suffering from cancer and wherein said method comprises the following steps:
a) Contacting said tissue sample with at least one hyperpolarized marker;
b) Obtaining an NMR spectrum and/or an MR image of said tissue sample;
c) Comparing said NMR spectrum and/or said MR image obtained in step b) and/or at least one parameter determined from said obtained NMR spectrum and/or MR image to a reference, wherein said reference corresponds to an NMR spectrum and/or an MR image of a healthy tissue sample and/or at least one parameter of a healthy tissue sample; and
d) Assigning cancer to said tissue sample based on the comparison carried out in step c), wherein a difference in the NMR spectrum and/or MR image and/or at least one parameter of said tissue sample and said reference is indicative of cancer.

Preferably, the above steps are not conducted on the human body; the above method thus corresponds to an in vitro method of diagnosis.

One could also make reference to the above method as an intra-operative method of non-invasively diagnosing cancer in a tissue sample since the steps of the above method result in the tissue sample remaining substantially intact; thus, the tissue sample may e.g. be used for further post-operative histopathology.

It should be noted that the above method of diagnosing cancer is particularly suitable for a specific patient population, namely patients suffering from cancer, e.g. breast cancer, and literally undergoing surgery for said cancer. Preferably, said patients are undergoing surgery for a primary tumor, such as e.g. a primary breast cancer tumor in the breast. The above method allows for the diagnosis of cancer in an excised tissue sample from said patients, e.g. in a sentinel lymph node, while the cancer surgery, e.g. for a primary breast cancer tumor, is still ongoing.

Said at least one hyperpolarized marker contains at least one NMR active nucleus, i.e. a nucleus with non-zero spin, preferably with spin ½, such as ¹H, ¹³C, ¹⁵N, ¹⁹F or ³¹P. The marker may be isotopically enriched. Preferably, said marker is isotopically enriched with ¹³C and/or ¹⁵N. Said marker containing at least one NMR active nucleus may be selected from the group consisting of fatty acids, amino acids, keto acids, TCA cycle intermediates, urea cycle intermediates, N-acetyl derivates of amino acids, carbohydrates, 2-amino-phosphono-carboxylic acids and fluorinated alpha amino acids, quaternary nitrogen containing compounds, salts thereof, esters thereof and mixtures thereof. More preferably, said at least one hyperpolarized marker contains at least one NMR active nucleus and is selected from the group consisting of acetate, acetoacetate, alanine, 2-oxoglutarate, arginine, asparagine, aspartate, beta-alanine, trimethylglycine, bicarbonate, butyrate, choline, cis-aconitic acid, creatine, cysteate, cysteine, fructose, fumarate, glucose, glutamate, glutamine, glycine, glyoxylic acid, guanidinoacetic acids, homocysteine, 4-hydroxyproline, 3-hydroxybutyrate, hydroxypyruvate, 2-ketoisocaproic acid, lactic acid, malic acid, methionine, N-acetyl aspartate, N-acetyl cysteine, oxaloacetate, phenylalanine, phenylpyruvate, proline, pyruvate, serine, taurine, ureidopropionate, isotopically enriched compounds thereof, salts thereof, esters thereof and mixtures thereof. Preferred esters are methyl esters or ethyl esters, in particular mono- and di-methyl esters and mono- and di-ethyl esters.

It can be preferred to use specific hyperpolarized markers for specific cancers, as will be explained in the following.

Generally, almost all cancer types have a higher energy turnover compared to healthy tissue surrounding the tumor. This can be exemplified with a higher glucose consumption leading to a larger lactate concentration in cancer cells, due to the Warburg effect. Hyperpolarized glucose containing at least one NMR active nucleus can thus e.g. be employed to assess the higher glycolytic rate. Also, a fast equilibrium between lactate and pyruvate makes it possible to employ e.g. hyperpolarized pyruvate containing at least one NMR active nucleus to visualize the increased pool of lactate.

Although its etiology is lacking, cancer is well characterized as a disease based on molecular aberrations. Thus, an altered activity of certain types of enzymes appears to be a general molecular alteration shared by different types of cancer cells. If a decrease of the enzymatic activity is observed, this may be due to a lower expression of the corresponding enzyme in cancer cells. One such particular family of enzymes that show a change in the activity (and in most cases also in the expression) and that may therefore be used as indicator for cancer (facilitating a “molecular fingerprint” of cancer) are carboxyl esterases. Recent reports link a lower activity (in some cases apparently based on a lower expression level) of carboxyl esterases with the presence of cancer (see e.g. Na, K. et al., Human plasma carboxylesterase 1, a novel serologic biomarker candidate for hepatocellular carcinoma (2009), Proteomics, 9: 3989-99 and Jansen et al., CPT-11 in human colon cancer cell lines and xenografts: characterization of cellular sensitivity determinants, 1997, Int. J. Cancer 70:335-40). Carboxyl esterases (CE) comprise a multigene family capable of hydrolyzing a large variety of carboxylic acid esters. The majority of CE isozymes belong to the CE1 and CE2 families and are differentiated on the basis of substrate specificity and tissue distribution. Preferentially, CE1 isozymes hydrolyse compounds esterified with a small alcohol group whereas CE2 isozymes hydrolyze compounds with a relatively small acyl group and a large alcohol group.

Hyperpolarized ester compounds containing at least one NMR active nucleus may thus be employed to assess the CE activity; if the CE activity in an analyzed tissue sample is substantially lower than in a reference sample of healthy tissue, such a lower CE-activity is indicative for the presence of cancer in the analyzed tissue.

The at least one NMR active nucleus in the ester compounds is preferably at least one ¹³C carbon atom (such that the compound may be referred to as “hyperpolarized ¹³C ester”), which may be part of the molecular moieties comprised in an ester, namely the acid, the alcohol or both. Therefore, the detected metabolic product after hydrolyzation of the ester may be the resulting acid, the resulting alcohol or both. If the ester used is ethyl acetoacetate, a corresponding metabolic product to be detected will e.g. be the acetoacetate anion.

A hyperpolarized ¹³C ester is preferably a mono- or dimethyl ester or a mono- or diethyl ester. Particularly suitable monoethyl esters are low molecular weight mono ethyl esters; particularly preferred monoethyl esters are ethyl acetoacetate and ethyl butyrate, with ethyl acetoacetate (such as e.g. 1,3-¹³C-ethyl acetoacetate) being most preferred. Suitable diethyl esters are diethyl succinate and diethyl 2-oxoglutarate, with diethyl succinate being most preferred.

Esters as discussed in the above section may be used as hyperpolarized markers for all types of cancer due to the correlation of lower CE-activity in cancer cells compared to healthy cells.

For specific cancer types, more specific alterations in the metabolite concentrations are known from the literature. For breast cancer, such alterations include the concentration of e.g. choline, creatine, glycine and taurine. Corresponding hyperpolarized markers that can assess these altered concentrations (by means of uptake and/or metabolism) are thus e.g. choline, guanidinoacetate, serine and cysteate containing at least one NMR active nucleus. For prostate cancer, other metabolic concentrations are specifically altered such as e.g. the concentration of aspartate, glutamine, glutamate, choline and branched chain amino acids. For these metabolites, the corresponding hyperpolarized markers that can assess the altered concentrations (by means of uptake and/or metabolism) are thus e.g. oxaloacetate, glutamate, 2-oxoglutarate, choline and 2-keto isocaproic acid containing at least one NMR active nucleus. Colon cancer exhibits high concentrations in the metabolites beta-alanine, asparagin, cysteine, methionine, phenylalanine, aspartate and butyrate. Hyperpolarized markers that can assess these altered concentrations (by means of uptake and/or metabolism) are thus e.g. ureidopropionate, asparate, N-acetylcysteine, homocysteine, phenylpyruvate, N-acetylaspartate and butyrate containing at least one NMR active nucleus.

A particularly preferred hyperpolarized marker for use in breast cancer diagnosis is 1,4-¹³C₂-fumarate.

In another preferred embodiment, said tissue sample is contacted in step a) with said at least one hyperpolarized marker by injection, e.g. through a needle or canula, and/or perfusion, e.g. accomplished by soaking the tissue in liquid containing the hyperpolarized marker and optionally increasing the perfusion by cycles of gentle compression/release of the tissue.

The contacting is preferably performed in a vessel where the tissue is embedded in a buffer, e.g. a physiological buffer (see also below description of optional further steps).

The cancer referred to in the present method may be breast cancer, prostate cancer, colon cancer, melanoma, ovarian cancer, head and neck cancer and gastric cancer. In a preferred embodiment, said cancer is selected from breast cancer, prostate cancer and colon cancer. Most preferably, said cancer is breast cancer.

Said tissue sample may correspond to rim tissue surrounding the excised primary tumor. Thus, depending on the cancer type as stated above, said tissue sample may be selected from the group consisting of breast tissue, prostate tissue, colon tissue, skin tissue, ovarian tissue, head and neck tissue and gastric tissue. If the patient suffers from breast cancer, the tissue sample may e.g. be breast tissue suspicious of corresponding to or comprising malignant tissue, preferably rim tissue surrounding the excised primary breast tumor in a breast.

In another preferred embodiment, said tissue sample is a lymph node tissue sample. It can be especially preferred that said tissue sample is a sentinel lymph node tissue sample. A lymph node is particularly preferred as tissue sample if the patient suffers from breast cancer, prostate cancer or colon cancer.

In another preferred embodiment, a cancer metastasis is diagnosed in said tissue sample. Thus, if the patient suffers e.g. from breast cancer, a breast cancer metastasis is diagnosed in said tissue sample, which may e.g. be a sentinel lymph node.

In a particularly preferred embodiment, said hyperpolarized marker is a marker taken up by cells and said difference in the NMR spectrum and/or MR image and/or said at least one parameter of said tissue sample and the reference is caused by an increased or decreased uptake of said marker by cancer cells compared to healthy cells, preferably an increased uptake. A particularly preferred parameter of said tissue sample, which may be determined from said obtained NMR spectrum and/or MR image, corresponds to an intracellular concentration of said marker. Said concentration may then be compared to a concentration of such a marker in healthy cells. Depending on the type of cancer and the marker taken up by the cells in the tissue, an increase or a decrease of the concentration is then indicative of cancer.

A hyperpolarized marker, which may be used as marker taken up by cells can be selected from the group consisting of compounds with quaternary nitrogen and/or long T₁ carbon containing markers, such as preferably ¹⁵N choline, ¹⁵N trimethylglycine, 1-¹³C acetate, 1-¹³C butyrate, 1-¹³C beta-alanine and 1- or 2-¹³C,²H₂ taurine, salts thereof, esters thereof and mixtures thereof. Particularly preferred markers for uptake are ¹⁵N choline and 1-¹³C acetate.

In another particularly preferred embodiment, said hyperpolarized marker is a pH sensitive marker and said difference in the NMR spectrum and/or MR image and/or said at least one parameter of said tissue sample and the reference is caused by a pH increase or decrease in cancer tissue compared to healthy tissue, preferably a pH decrease. A particularly preferred parameter of said tissue sample, which may be determined from said obtained NMR spectrum and/or MR image, corresponds to the pH value of said tissue sample. Said pH value may then be compared to the pH value of healthy tissue. Usually, cancer tissue has a lower pH value than healthy cells.

A hyperpolarized marker, which may be used as pH sensitive marker can be selected from the group consisting of ¹³C bicarbonate, 2-amino-phosphono-carboxylic acids (³¹P detection), fluorinated alpha amino acids (¹⁹F detection), salts thereof, esters thereof and mixtures thereof. A preferred fluorinated alpha amino acid is ¹⁹F difluoromethylalanine. A particularly preferred pH sensitive marker is ¹³C bicarbonate.

In yet another particularly preferred embodiment, said hyperpolarized marker is a metabolic marker and said difference in the NMR spectrum and/or MR image and/or said at least one parameter of said tissue sample and the reference is caused by an altered metabolic profile in cancer tissue compared to healthy tissue, preferably an increased metabolite concentration, up-regulated enzyme expression, higher enzymatic activity higher co-substrate concentration or mixtures thereof. A particularly preferred parameter of said tissue sample, which may be determined from said obtained NMR spectrum and/or MR image, corresponds to the concentration of a metabolite of said marker. Said concentration may then be compared to a concentration of such a metabolite in healthy tissue. Depending on the type of cancer and the metabolic marker, an increase or a decrease of the concentration of the metabolite is then indicative of cancer.

A hyperpolarized marker, which may be used as metabolic marker contains at least one NMR active nucleus and can be selected from the group consisting of amino acids, keto acids, TCA cycle intermediates, urea cycle intermediates such as acetate, acetoacetate, alanine, 2-oxoglutarate, arginine, asparagine, aspartate, bicarbonate, butyrate, cis-aconitic acid, creatine, cysteate, cysteine, fructose, fumarate, glucose, glutamate, glutamine, glycine, glyoxylic acid, guanidinoacetic acid, homocysteine, 4-hydroxyproline, 3-hydroxybutyrate, hydroxypyruvate, 2-ketoisocaproic acid, lactic acid, malic acid, methionine, N-acetyl aspartate, N-acetyl cysteine, oxaloacetate, phenylalanine, phenylpyruvate, proline, pyruvate, serine, ureidopropionate, isotopically enriched compounds thereof, salts thereof, esters thereof (e.g methyl esters or ethyl esters) and mixtures thereof. A particularly preferred metabolic marker contains at least one NMR active nucleus and is selected from the group consisting of acetoacetate, 2-oxoglutarate, aspartate, fumarate, glucose, glutamine, 3-hydroxybutyrate, 2-ketoisocaproic acid, pyruvate, isotopically enriched compounds thereof, salts thereof, esters thereof and mixtures thereof, all of which contain at least one NMR active nucleus.

Further hyperpolarized markers, which may be used as metabolic markers, are the hyperpolarized ester compounds containing at least one NMR active nucleus as discussed above, in particular mono- and di-methy esters and mono- and di-ethyl esters, wherein ethyl acetoacetate is most preferred as mono-ethyl ester.

Another preferred embodiment refers to the above method comprising at least one further step preceding step a), namely
Removing non-adherent cells from said tissue sample.
   Such a step may increase sensitivity since the tissue sample to be analyzed substantially only comprises adherent cells following this step. Further, this step may correspond to a partial preparation of the tissue sample for a histopathology carried out subsequent to the present method.

Another preferred embodiment refers to the above method comprising at least one further step preceding step a), namely
Transferring the tissue to a vessel.
   Such a step may facilitate different means of contacting the tissue e.g. by adding liquid in the form of a buffer. Also, the vessel may then be directly used in the MR scanner. Said tissue sample may be transferred to a vessel by the use of e.g. forceps or the like. Generally, said tissue sample is preferably transferred to said vessel immediately (i.e. within a time period of up to 300 seconds, preferably up to 120 seconds, more preferably up to 60 seconds, and most preferably within 30 seconds) after having been obtained.

Another preferred embodiment refers to the above method comprising at least one further step preceding step a), namely
Contacting said tissue sample with a buffer, preferably a physiological buffer.
   In doing so, the tissue may already partially be prepared for a subsequent histopathology; further, said step may preserve the cells for said histopathology, particularly if a physiological buffer such as e.g. Ringer's solution is used. Such a physiological buffer may have a temperature of 37° C.

After the transfer to a vessel, the tissue sample is preferably immediately (i.e. within a time period of up to 60 seconds, preferably up to 30 seconds, more preferably up to 20 seconds, and most preferably up to 10 or up to 5 seconds) contacted with buffer. Preferably, the volume of the buffer is large enough to cover the tissue sample and, typically, 0.1 to 10 ml buffer, preferably 0.2 to 2 ml are used.

It should be mentioned that the above listed steps may be carried out in combination. If e.g. the just-mentioned Transferring and Contacting steps are carried out, the obtained tissue sample is present in buffer in a vessel suitable for use in an MR device.

Another preferred embodiment refers to the above method comprising at least one further step preceding step a), namely
Contacting said tissue sample with at least one substance selected from the group consisting of an unlabeled co-substrate; an inhibitor specifically blocking the uptake of certain substances into healthy cells; a substance decreasing or increasing the pH; and a substance specifically increasing enzymatic activity in cancer cells compared to healthy cells.

Said additional step of contacting said tissue sample with at least one of the above listed substances prior to contacting the tissue sample with said at least one hyperpolarized marker is particularly suitable if one of the above described embodiments using specific markers is carried out. The above listed substances are preferably incubated with the tissue sample for a time period, which is in the range of 1 second to 10 minutes. In this respect, it should be emphasized that the present method corresponds to an in vitro method such that the above listed substances may indeed be employed in order to increase specificity and sensitivity of the method, contrary to an in vivo method, wherein such substances might even be toxic. Preferably, the addition of at least one of the above listed substances fails to interfere with an independent histopathology or any other state of the art diagnostic method carried out subsequent to the present method.

The specificity of the uptake of at least one hyperpolarized marker into cancerous cells may be increased by the addition of an inhibitor specifically blocking the uptake of specific substances into healthy cells; in doing so, said marker is specifically taken up by cancerous cells only. Such inhibitors may particularly be used in combination with a marker taken up by cells; however, specificity may also be increased if a metabolic marker is used. An exemplary compound in this respect is p-Chloromercuribenzoic acid (pCMBS), which inhibits the monocarboxylic transporters 1 and 4 (MCT1 and MCT4, which are active in both normal and cancerous cells), whereas it does not inhibit MCT 2 (which is only active in cancer cells). Typically, pCMBS is added to a final concentration of 1 mM.

By adding a substance decreasing or increasing the pH, the uptake and/or the metabolism may be altered in the cells of the analyzed tissue sample. Due to the existing differences between healthy cells and cancerous cells, altering the pH may introduce further specificity for discriminating between healthy cells and cancerous cells (e.g. by taking advantage of an increased enzyme expression in the cancer cells compared to the healthy cells by eliminating a rate limiting uptake of the marker). Such a substance is preferably selected from a group of acidic or basic buffers (e.g. citric acid, formic acid, acetic acid and ammonium), the choice of which will depend on the pKa of the marker. A change of the pH environment in the tissue sample may particularly be used in combination with a metabolic marker.

An unlabeled co-substrate may particularly be added if a hyperpolarized metabolic marker is used; generally, said co-substrate is chosen depending on the metabolic marker used, i.e. depending on the metabolic reaction that the marker participates in. If e.g. the metabolic marker 1-¹³C ketoisocaproic acid is used, the co-substrate may e.g. be glutamate.

By adding a substance specifically increasing enzymatic activity in cancer cells compared to healthy cells, the specificity and sensitivity of the diagnosis may also be increased. Typically, such a substance corresponds to a substrate or co-substrate of an enzyme which is overexpressed in cancerous cells compared to healthy cells and which is part of a metabolic pathway. Preferably, such a substance is used in combination with a hyperpolarized metabolic marker. It is selected depending on the metabolic pathway analyzed and the expression level of enzymes in this pathway in cancerous cells. If the metabolic marker is e.g. 5-¹³C glutamine acid and thus involved in the glutamate synthesis pathway, said substance may be phosphate since it activates the enzyme glutaminase 1 which is overexpressed in cancer cells.

Another preferred embodiment refers to the above method comprising at least one further step preceding step a), namely
Tempering said tissue sample.

By tempering the tissue sample to a specific temperature or a temperature range, the uptake and/or the metabolism may be altered in the cells of the analyzed tissue sample. Due to the existing differences between healthy cells and cancerous cells, specific temperature ranges or temperatures may introduce further specificity for discriminating between healthy cells and cancerous cells. Thus, it can be preferred to increase the temperature to a temperature range or a specific temperature above room temperature, e.g. to a range of between 30° C. and 40° C. or e.g. to 37° C. For certain analyses, it might be preferred to lower the temperate to a temperature range or temperature below room temperature, e.g. to a range of between 4° C. and 15° C. or e.g. to 10° C. Specific temperature ranges or temperatures may be achieved by corresponding means known to the skilled person, e.g. the spectrometer device, heat-blocks, cooling agents (such as ice-baths), and the like. Of course, room temperature may also be preferred in certain embodiments.

Another preferred embodiment refers to the above method comprising at least one further step between step a) and step b), namely
Contacting said tissue sample with at least one paramagnetic relaxation agent resulting in the loss of hyperpolarization of said at least one marker in the extracellular compartment.

A paramagnetic relaxation agent may particularly be added if a hyperpolarized marker taken up by cells is used. Using a paramagnetic relaxation agent, the uptake may be determined more specifically since no signals from markers outside of cells are detected any more. The paramagnetic relaxation agent may be selected from the group consisting of paramagnetic metal ions and complexes thereof. A preferred paramagnetic relaxation agent is Omniscan (product of GE Healthcare).

The incubation period before the contacting step with at least one paramagnetic relaxation agent (i.e. the incubation period after completion of step a)) is usually about 1 second to about 10 minutes, depending on the marker and the NMR active nucleus. If ¹⁵N is used, the incubation period is preferably about 1 to about 5 minutes, more preferably 1 minute, or 2 minutes, or 3 minutes. If ¹³C is used, the incubation period is preferably about 5 seconds to about 60 seconds, more preferably 10 seconds, or 20 seconds, or 30 seconds, or 40 seconds.

The tissue sample may then be incubated with the at least one relaxing agent for about 1 second to about 30 seconds, preferably 1 second to 5 seconds, more preferably 1 second, or 2 seconds, or 3 seconds.

In a particularly preferred embodiment, said hyperpolarized marker is hyperpolarized by dynamic nuclear polarization (DNP).

In a preferred embodiment of the first object, the present invention relates to a method of diagnosing a cancer metastasis in a lymph node tissue sample, wherein said method is carried out on an excised lymph node tissue sample obtained from a patient suffering from cancer and wherein said method comprises the following steps:
a) Contacting said tissue sample with a combination of (i) a hyperpolarized metabolic marker indicating metabolically active cells and (ii) a hyperpolarized metabolic marker allowing a distinction between lymphocytes and cancer cells;
b) Obtaining an NMR spectrum and/or an MR image of the metabolic products of (i) and (ii) comprised in said tissue sample;
c) Determining the ratio r_{S} of the metabolic products of (i) to the metabolic products of (ii);
d) Comparing the ratio r_{S} obtained in step c) to a reference ratio r_{R} of the metabolic products of (i) to the metabolic products of (ii) obtained in a lymph node tissue sample consisting of healthy cells; and
e) Assigning cancer to said tissue sample based on the comparison carried out in step d), wherein a difference in the ratio r_{S} to the ratio r_{R} indicates the presence of a cancer metastasis in said tissue sample.

In a preferred embodiment relating to the above method, (ii) is a carboxylate ester of a molecular weight of ≦400 Da, preferably a stable and soluble carboxylate ester of a molecular weight of ≦400 Da, more preferably a stable and soluble carboxylate ester of a molecular weight of ≦400 Da comprising a short alkyl-chain in the alcohol position and a short acid part. Preferably, said short alkyl-chain is methyl, ethyl, propyl, butyl, pentyl or hexyl (wherein the propyl-, butyl-, pentyl- or hexyl-chain is optionally branched) or benzyl. It can further be preferred that the short acid part comprises up to five optionally branched carbon units. It should be noted that the above does not exclude esters of di-acids or diols or triols; however, the indications for the alcohol-part(s) and the acid part(s) as given above then apply accordingly to these molecules.

It is preferred that esters of ring closed acids (such as esters of pyroglutamate), esters of tri-acids (such as esters of citrate) and ethylacetate are excluded from the ester used herein.

A preferred carboxylate ester is an ester selected from esters of acetic acid with a molecular weight of ≦400 Da, wherein the alcohol part comprises a straight or branched alkyl chain and/or aromatic group, wherein said straight or branched alkyl chain and/or aromatic group is optionally functionalized, and from ethyl or methyl esters of a carboxylic acid with a molecular weight of ≦400 Da, wherein the acid part comprises a straight or branched alkyl chain and/or aromatic group, wherein said straight or branched alkyl chain and/or aromatic group is optionally functionalized.

The ester may be an ester of an unsubstituted acid (such as acetate or butyrate), an ester of a C2-substituted acid (such as lactate or 2-acetoxy propanoate), an ester of a C3-substituted acid (such as 3-hydroxy butyrate or 3-acetoxy butanoate), an ester of a di-acid (such as succinate) or an ester of a beta-keto acid (such as acetoacetate). The alcohol part of the ester may be a diol (such as ethylen glycol) or a triol (such as glycerol), in combination with a short acid part (such as acetate, propanoate or butyrate).

Particularly preferred esters are esters of di-acids, particularly dimethyl-, diethyl-, dipropyl- and dibutyl-esters with an acid part comprising up to five optionally branched carbon units; esters of mono-acids, particularly methyl-, ethyl-, propyl- and butyl-esters with an acid part comprising up to five optionally branched carbon units; aromatic esters with an acid part comprising up to five optionally branched carbon units, particularly optionally substituted benzyl acetate; and esters derived from diols and triols with an alcohol part comprising up to five optionally branched carbon units and an acid part comprising up to five optionally branched carbon units, particularly ethyleneglycol diacetate and triacetin.

Examples of particularly preferred esters are the following: ethyl butyrate, metyl butyrate, ethyl lactate, ethyl 2-acetoxy propanoate, ethyl 3-hydroxy butyrate, ethyl 3-acetoxy butanoate, dietyl succinate, dimethyl succinate, ethyl acetoacetate, ethyl 3-acetoxy butanoate, ethyl 2-acetoxy propionate, benzyl acetate, ethyleneglycol diacetate, triacetin and ethyl 3-acetoxybutanoate.

In another preferred embodiment of the first object, the present invention relates to a method of diagnosing a cancer metastasis in a lymph node tissue sample, wherein said method is carried out on an excised lymph node tissue sample obtained from a patient suffering from cancer and wherein said method comprises the following steps:
a) Contacting said tissue sample with a combination of (i) a hyperpolarized metabolic marker indicating metabolically active cells and (ii) a hyperpolarized metabolic marker exhibiting either a higher conversion in cancer cells compared to lymphocytes or a lower conversion in cancer cells compared to lymphocytes;
b) Obtaining an NMR spectrum and/or an MR image of the metabolic products of (i) and (ii) comprised in said tissue sample;
c) Determining the ratio r_{S} of the metabolic products of (i) to the metabolic products of (ii);
d) Comparing the ratio r_{S} obtained in step c) to a reference ratio r_{R} of the metabolic products of (i) and (ii) obtained in a lymph node tissue sample consisting of healthy cells; and
e) Assigning cancer to said tissue sample based on the comparison carried out in step d), wherein a ratio of r_{S}:r_{R} of >1 indicates the presence of a cancer metastasis in said tissue sample if (ii) exhibits a higher conversion in cancer cells compared to lymphocytes or wherein a ratio of r_{R}:r_{S} of >1 indicates the presence of a cancer metastasis in said tissue sample if (ii) exhibits a lower conversion in cancer cells compared to lymphocytes.

It can be preferred that the metabolic marker exhibiting a higher conversion rate in cancer cells compared to lymphocytes is an ester, which is a substrate for carboxyl esterase 2. The alcohol part of the ester may be a diol (such as ethylen glycol) or a triol (such as glycerol), in combination with a short acid part (such as acetate, propanoate or butyrate). Particularly preferred esters are aromatic esters with an acid part comprising up to five optionally branched carbon units, particularly optionally substituted benzyl acetate; and esters derived from diols and triols with an alcohol part comprising up to five optionally branched carbon units and an acid part comprising up to five optionally branched carbon units, particularly ethyleneglycol diacetate and triacetin. Particularly preferred esters can be butyl acetate, t-butyl acetate, ethyl 3-acetoxy butanoate, 2-acetoxy propionate, benzyl acetate, ethyleneglycol diacetate, triacetin and ethyl 3-acetoxybutanoate. An especially preferred ester is selected from the group consisting of acetin, benzyl acetate and ethyl 3-acetoxybutanoate. The ester may also be selected from esters of acetic acid with a molecular weight of ≦400 Da, wherein the alcohol part comprises a straight or branched alkyl chain and/or aromatic group, wherein said straight or branched alkyl chain and/or aromatic group is optionally functionalized.

It can be preferred that the metabolic marker exhibiting a lower conversion rate in cancer cells compared to lymphocytes is an ester, which is a substrate for carboxyl esterase 1. The ester may be an ester of an unsubstituted acid (such as acetate or butyrate), an ester of a C2-substituted acid (such as lactate or 2-acetoxy propanoate), an ester of a C3-substituted acid (such as 3-hydroxy butyrate or 3-acetoxy butanoate), an ester of a di-acid (such as succinate) or an ester of a beta-keto acid (such as acetoacetate). Particularly preferred esters are esters of di-acids, particularly dimethyl-, diethyl-, dipropyl- and dibutyl-esters with an acid part comprising up to five optionally branched carbon units; and esters of mono-acids, particularly methyl-, ethyl-, propyl- and butyl-esters with an acid part comprising up to five optionally branched carbon units. Particularly preferred esters can be ethyl butyrate, metyl butyrate, ethyl lactate, ethyl 2-acetoxy propanoate, ethyl 3-hydroxy butyrate, ethyl 3-acetoxy butanoate, dietyl succinate, dimethyl succinate and ethyl acetoacetate. An especially preferred ester is selected from the group consisting of diethyl succinate, methyl butyrate and ethyl acetoacetate. The ester may also be selected from ethyl or methyl esters of a carboxylic acid with a molecular weight of ≦400 Da, wherein the acid part comprises a straight or branched alkyl chain and/or aromatic group, wherein said straight or branched alkyl chain and/or aromatic group is optionally functionalized.

In a particularly preferred embodiment of the first object, the present invention relates to a method of diagnosing a cancer metastasis in a lymph node tissue sample, wherein said method is carried out on an excised lymph node tissue sample obtained from a patient suffering from breast cancer and wherein said method comprises the following steps:
a) Contacting said tissue sample with a combination of (i) a hyperpolarized metabolic marker indicating metabolically active cells and (ii) a hyperpolarized metabolic marker exhibiting a lower conversion in cancer cells compared to lymphocytes;
b) Obtaining an NMR spectrum and/or an MR image of the metabolic products of (i) and (ii) comprised in said tissue sample;
c) Determining the ratio r_{S} of the metabolic products of (i) to the metabolic products of (ii);
d) Comparing the ratio r_{S} obtained in step c) to a reference ratio r_{R} of the metabolic products of (i) and (ii) obtained in a lymph node tissue sample consisting of healthy cells; and
e) Assigning cancer to said tissue sample based on the comparison carried out in step d), wherein a ratio of r_{R}:r_{S} of >1 indicates the presence of a cancer metastasis in said tissue sample.

It can be preferred that the metabolic marker exhibiting a lower conversion rate in breast cancer cells compared to lymphocytes is an ester, which is a substrate for carboxyl esterase 1. Such esters have been described above, wherein diethyl succinate is most preferred as metabolic marker exhibiting a lower conversion rate in breast cancer cells compared to lymphocytes.

In another particularly preferred embodiment of the first object, the present invention relates to a method of diagnosing a cancer metastasis in a lymph node tissue sample, wherein said method is carried out on an excised lymph node tissue sample obtained from a patient suffering from prostate cancer and wherein said method comprises the following steps:
a) Contacting said tissue sample with a combination of (i) a hyperpolarized metabolic marker indicating metabolically active cells and (ii) a hyperpolarized metabolic marker exhibiting a higher conversion in cancer cells compared to lymphocytes;
b) Obtaining an NMR spectrum and/or an MR image of the metabolic products of (i) and (ii) comprised in said tissue sample;
c) Determining the ratio r_{S} of the metabolic products of (i) to the metabolic products of (ii);
d) Comparing the ratio r_{S} obtained in step c) to a reference ratio r_{R} of the metabolic products of (i) and (ii) obtained in a lymph node tissue sample consisting of healthy cells; and
e) Assigning cancer to said tissue sample based on the comparison carried out in step d), wherein a ratio of r_{S}:r_{R} of >1 indicates the presence of a cancer metastasis in said tissue sample.

It can be preferred that the metabolic marker exhibiting a higher conversion rate in prostate cancer cells compared to lymphocytes is an ester, which is a substrate for carboxyl esterase 2. Such esters have been described above, wherein benzyl acetate is most preferred as metabolic marker exhibiting a higher conversion rate in prostate cancer cells compared to lymphocytes.

In a preferred embodiment relating to step e) of the above methods, a ratio of r_{S}:r_{R} or of r_{R}:r_{S} of >1.5, preferably of >2, more preferably of >3 and most preferably of >5 indicates the presence of a cancer metastasis in said tissue sample.

In a preferred embodiment relating to all of the above methods, said metabolic marker indicating metabolically active cells is selected from the group consisting of glucose, pyruvate, lactate, fumarate, malate, an alpha-keto acid and an alpha amino acid. The alpha-keto acid is preferably selected from 2-ketoisocaproic acid and 2-oxoglutarate. The alpha amino acid is preferably selected from glutamate and aspartate.

It can be preferred that said metabolic marker indicating metabolically active cells is selected from glucose and pyruvate, wherein ¹³C₆-d₇-glucose and 1-¹³C-pyruvate are particularly preferred.

As indicated also further below, all of the metabolic markers referred to above contain at least one NMR active nucleus, wherein ¹³C is preferred, and are further preferably isotopically enriched with said at least one NMR active nucleus, preferably ¹³C. Said NMR active nucleus may in principle be at any position in the metabolic marker, wherein long T₁ ¹³C-positions (>10 s at 3 T and 37° C.) of the metabolic markers are most preferred for the methods according to the present invention.

As concerns the above methods of diagnosing a cancer metastasis, the above steps are preferably not conducted on the human body; the above method thus corresponds to an in vitro method of diagnosis. One could also make reference to the above method as an intra-operative method of non-invasively diagnosing a cancer metastasis in a lymph node tissue sample since the steps of the above method result in the tissue sample remaining substantially intact; thus, the tissue sample may e.g. be used for further post-operative histopathology. It should be noted that the above method is particularly suitable for a specific patient population, namely patients suffering from cancer, e.g. breast cancer, and literally undergoing surgery for a primary tumor, such as e.g. a primary breast cancer tumor in the breast. The above method allows for the diagnosis of a cancer metastasis in an excised lymph node tissue sample from said patients while the cancer surgery, e.g. for a primary breast cancer tumor, is still ongoing.

The at least one further optional step as disclosed in general above of course also applies for the above methods of diagnosing a cancer metastasis.

In a second object, the present invention is concerned with the use of a method as outlined above for providing a diagnosis of cancer in a tissue sample while the patient is undergoing a cancer surgery. In a preferred embodiment, this relates to the use in the diagnosis of a cancer metastasis in a sentinel lymph node as tissue sample obtained from a patient suffering from breast cancer, prostate cancer, head and neck cancer or colon cancer undergoing a cancer surgery. The present method may thus be used for the decision-making on further surgical steps during the ongoing surgery.

Further, the present invention relates to the use of a hyperpolarized marker in an in vitro method of intra-operatively diagnosing cancer.

In a third object, the present invention relates to a combination or kit comprising (i) a metabolic marker indicating metabolically active cells and (ii) a metabolic marker allowing a distinction between lymphocytes and cancer cells. It is preferred that said metabolic markers (i) and (ii) are the only metabolic markers comprised in said combination or kit. Said metabolic markers (i) and (ii) may be present in said combination or kit in a solid state or dissolved in a suitable solvent in liquid state. The combinations as disclosed and claimed herein are preferably hyperpolarized and then used as markers in a method as set out above. A combination comprising the metabolic markers as set out in the following is thus deemed to also encompass a combination of corresponding hyperpolarized metabolic markers.

In a preferred embodiment relating to the above combination or kit, (ii) is a carboxylate ester of a molecular weight of ≦400 Da, preferably a stable and soluble carboxylate ester of a molecular weight of ≦400 Da, more preferably a stable and soluble carboxylate ester of a molecular weight of ≦400 Da comprising a short alkyl-chain in the alcohol position and a short acid part. Preferably, said short alkyl-chain is methyl, ethyl, propyl, butyl, pentyl or hexyl (wherein the propyl-, butyl-, pentyl- or hexyl-chain is optionally branched) or benzyl. It can further be preferred that the short acid part comprises up to five optionally branched carbon units. It should be noted that the above does not exclude esters of di-acids or diols or triols; however, the indications for the alcohol-part(s) and the acid part(s) as given above then apply accordingly to these molecules. It is preferred that esters of ring closed acids (such as esters of pyroglutamate), esters of tri-acids (such as esters of citrate) and ethylacetate are excluded from the ester referred to under (ii) above.

A preferred metabolic marker (ii) of the combination or kit according to the invention is an ester selected from esters of acetic acid with a molecular weight of ≦400 Da, wherein the alcohol part comprises a straight or branched alkyl chain and/or aromatic group, wherein said straight or branched alkyl chain and/or aromatic group is optionally functionalized, and from ethyl or methyl esters of a carboxylic acid with a molecular weight of ≦400 Da, wherein the acid part comprises a straight or branched alkyl chain and/or aromatic group, wherein said straight or branched alkyl chain and/or aromatic group is optionally functionalized.

An ester as metabolic marker (ii) as comprised in a combination or kit according to the present invention may be an ester of an unsubstituted acid (such as acetate or butyrate), an ester of a C2-substituted acid (such as lactate or 2-acetoxy propanoate), an ester of a C3-substituted acid (such as 3-hydroxy butyrate or 3-acetoxy butanoate), an ester of a di-acid (such as succinate) or an ester of a beta-keto acid (such as acetoacetate). The alcohol part of the ester may be a diol (such as ethylen glycol) or a triol (such as glycerol), in combination with a short acid part (such as acetate, propanoate or butyrate).

Particularly preferred esters as metabolic markers (ii) as comprised in a combination or kit according to the present invention are esters of di-acids, particularly dimethyl-, diethyl-, dipropyl- and dibutyl-esters with an acid part comprising up to five optionally branched carbon units; esters of mono-acids, particularly methyl-, ethyl-, propyl- and butyl-esters with an acid part comprising up to five optionally branched carbon units; aromatic esters with an acid part comprising up to five optionally branched carbon units, particularly optionally substituted benzyl acetate; and esters derived from diols and triols with an alcohol part comprising up to five optionally branched carbon units and an acid part comprising up to five optionally branched carbon units, particularly ethyleneglycol diacetate and triacetin. Examples of particularly preferred esters are the following: ethyl butyrate, metyl butyrate, ethyl lactate, ethyl 2-acetoxy propanoate, ethyl 3-hydroxy butyrate, ethyl 3-acetoxy butanoate, dietyl succinate, dimethyl succinate, ethyl acetoacetate, ethyl 3-acetoxy butanoate, ethyl 2-acetoxy propionate, benzyl acetate, ethyleneglycol diacetate, triacetin and ethyl 3-acetoxybutanoate.

Preferably, (ii) in the combination or kit according to the present invention is selected from the group consisting of acetin, benzyl acetate, ethyl 3-acetoxybutanoate, diethyl succinate, methyl butyrate and ethyl acetoacetate. More preferably, (ii) is selected from the group consisting of benzyl acetate, diethyl succinate and ethyl acetoacetate.

In another preferred embodiment, (i) as comprised in the combination or kit according to the present invention is selected from the group consisting of glucose, pyruvate, lactate, fumarate, malate, an alpha-keto acid and an alpha amino acid. The alpha-keto acid is preferably selected from 2-ketoisocaproic acid and 2-oxoglutarate. The alpha amino acid is preferably selected from glutamate and aspartate.

In a particularly preferred embodiment of said combination or kit, (i) is selected from the group consisting of glucose, pyruvate, lactate, fumarate, malate, 2-ketoisocaproic acid, 2-oxoglutarate, glutamate and aspartate and (ii) is selected from the group consisting of acetin, benzyl acetate, ethyl 3-acetoxybutanoate, diethyl succinate, methyl butyrate and ethyl acetoacetate.

In another preferred embodiment of the third aspect, (i) is selected from glucose and pyruvate and (ii) is selected from the group consisting of acetin, benzyl acetate, ethyl 3-acetoxybutanoate, diethyl succinate, methyl butyrate and ethyl acetoacetate.

In a preferred embodiment of the third aspect, (i) is selected from glucose and pyruvate and (ii) is selected from the group consisting of benzyl acetate, diethyl succinate and ethyl acetoacetate.

In a particularly preferred embodiment of the third aspect, (i) is pyruvate and (ii) is diethyl succinate or ethyl acetoacetate; or (i) is glucose and (ii) is benzyl acetate or ethyl acetoacetate.

All of the metabolic markers referred to in the third aspect of the present invention contain at least one NMR active nucleus, wherein ¹³C is preferred, and are further preferably isotopically enriched with said at least one NMR active nucleus, preferably ¹³C. Said NMR active nucleus may in principle be at any position in the metabolic marker, wherein long T₁ ¹³C-positions (>10 s at 3 T and 37° C.) of the metabolic markers are most preferred for the methods according to the present invention.

In a fourth object, the present invention relates to a diagnostic composition comprising any of the combinations as claimed and as outlined above in the third aspect.

The present invention also relates to the use of a combination, a kit and/or a diagnostic composition as disclosed and claimed herein for diagnosing a cancer metastasis in a lymph node tissue sample, preferably while the patient is undergoing a cancer surgery for a primary tumor, wherein said cancer is preferably selected from breast cancer, prostate cancer, head and neck cancer and colon cancer.

### DESCRIPTION OF THE FIGURES

FIG. 1: Metabolic conversion of hyperpolarized 1-¹³C pyruvate to 1-¹³C-lactate in a non-cancerous immortalized prostate cell line of human origin (PNT) and a prostate cancer cell line isolated from a metastasis of human origin (PC-3). A 1D ¹³C spectrum was obtained every 3 seconds using a 15 deg flip angle.

FIG. 2: Conversion of hyperpolarized 1,4-¹³C₂ fumarate to 1,4-¹³C₂ malate in a non-cancerous immortalized breast cell line (184B4) and in a breast cancer cell line (MDA-MB-231), both of human origin. The cells have been in contact with the hyperpolarized marker for 30 sec and the amount of the metabolite 1,4-¹³C₂ malate determined in each cell line was normalized to the amount of soluble protein.

FIG. 3: A) shows the change in the bicarbonate and carbondioxide ratio as a function of pH as calculated from the Henderson-Hasselbalch equation (see insert in A). B) depicts a ¹³C spectrum illustrating the separation of the two peaks corresponding to bicarbonate and carbondioxide. The ratio of the two integrals allows for a direct determination of the pH in the solution.

FIG. 4: Conversion of hyperpolarized 1,3-¹³C₂ ethyl acetoacetate in intact human breast cancer cells (MCF-7), human prostate cancer cells (PC-3) and human lymphocytes (20 million cells). A) Bar chart showing maximum hyperpolarized metabolite signal (1,3-¹³C₂ acetoacetate) in MCF-7, PC-3 and lymphocytes; B) Build-up of the metabolite, 1,3-¹³C₂ acetoacetate in MCF-7 (filled diamond), PC-3 (filled triangle) and in lymphocytes (filled circles).

FIG. 5: Bar chart of the hyperpolarized metabolic products [a.u] of ¹³C₆-d₇-glucose, 1-¹³C-pyruvate and 1,3-¹³C₂ ethyl acetoacetate in three different cell types scaled to the number of cells in a voxel volume of 8 μl: 2.6 million MCF-7 cancer cells, 1.1 million PC-3 cancer cells and 40 million lymphocytes.

FIG. 6: Ratio chart of 1-¹³C-lactate over 1,3-¹³C₂ acetoacetate in whole cell experiments corresponding to 2×2×2 voxels of 100%, 50% and 0% breast cancer cells (MCF-7) in the background of lymphocytes (Lymph.).

FIG. 7: Measured substrates grouped based on the measured CES1 (CE-1) activities linked to the structural properties of the substrates.

FIG. 8: CES2 (CE-2) activities of acetate esters grouped based on the measured CE-2 activities linked to the structural properties of the substrates. The activities are measured in 5 million lysed human breast cancer cells (MCF-7) and given in μmol/hour.

FIG. 9: Ratio chart for metabolic conversion of different esters in A) breast cancer cells relative to lymphocytes and in B) prostate cells relative to lymphocytes. Ratio (cancer/lymph) indicates a positive contrast (higher conversion in cancer cells compared to lymphocytes) whereas Ratio (lymph/cancer) indicates a negative contrast (lower conversion in cancer cells compared to lymphocytes).

### DETAILED DESCRIPTION OF THE INVENTION

The inventors of the present invention inter alia succeeded in providing a fast and reliable method of diagnosing cancer in vitro, which is carried out on a tissue sample obtained from a patient suffering from cancer and undergoing a surgery for said cancer. The method can be carried out intra-operatively and in a non-invasive manner.

The inventors further succeeded in providing a method, which is able to discriminate between cancer cells and lymphocytes when analyzing a lymph node tissue sample; this method is based on the use of a specific combination of hyperpolarized metabolic markers, namely (i) a metabolic marker indicating metabolically active cells and (ii) a metabolic marker allowing a distinction between lymphocytes and cancer cells. This method was surprisingly found before the background that (a) a lymph node sample comprises also non-metabolizing cells and (b) some metabolic markers do not seem to allow a distinction between lymphocytes and cancer cells to a sufficient degree.

Before some of the embodiments of the present invention are described in more detail, the following definitions are introduced.

### 1. Definitions

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “intra-operatively” is to be understood as referring to an ongoing surgery. Thus, the present method may also be referred to as method carried out during surgery or while the surgery is still ongoing, wherein the surgery itself is not part of the method. The method is preferably carried out within about 60 minutes, more preferably about 45 minutes, even more preferably about 30 minutes and most preferably in about 15 minutes to 20 minutes. Since an average cancer surgery takes about 60 to 90 minutes or about 120 minutes if the sentinel lymph node is removed, the present method can easily be performed within the average time frame for a surgery.

The term “diagnosing cancer/a cancer metastasis” is to be understood as “determining the presence or absence of cancer”. Thus, one may also refer to a method of intra-operatively determining the presence or absence of cancer/a cancer metastasis in a tissue sample. Accordingly, the term “assigning cancer” needs to be understood in the meaning of “assigning the presence or absence of cancer/a cancer metastasis”.

The term “tissue sample” refers to a sample obtained from a patient by surgery, thus also referred to as “excised” (wherein the step of surgery is not part of the method as claimed herein), wherein said sample comprises at least one cell, preferably at least one viable cell, from said patient and preferably corresponds to an accumulation of cells from the patient.

The term “viable” means that the cells are intact cells; it is not to be understood as referring either to healthy or cancerous. Rather, healthy as well as cancerous cells can be viable cells according to the present invention. The term “non-malignant” may be used instead of “healthy”; accordingly, “malignant” is used in the meaning of “cancerous”.

The term “transferring” as used herein refers to any suitable means of placing the obtained tissue sample after complete excision into a vessel; this may be done by surgical instruments such as e.g. sterile forceps or the like.

A “vessel” refers to any suitable means for holding the tissue sample. Any vessel suitable for holding the tissue sample, preferably in buffer, and for being loaded into and/or being used in an MR device is suitable for purposes of the present invention. Preferably, the vessel is able to carry a volume of 0.5 ml to 8 ml, most preferred about 1 ml.

The term “contacting” as used herein means that two objects are brought into direct physical contact, e.g. by pipetting the first object onto a second object, or by injecting a first object into a second object.

The term “marker” as used herein refers to a compound containing at least one NMR active nucleus, i.e. a nucleus with non-zero spin, preferably with spin ½, such as e.g. ¹H, ¹³C, ¹⁵N, ¹⁹F or ³¹P. The marker may be isotopically enriched, e.g. with ¹³C or ¹⁵N. The optimal position for isotopic enrichment in the marker is dependent on the relaxation time of the NMR active nucleus. Preferably, a marker is isotopically enriched in positions with long T₁ relaxation time. Preferably, such a marker is based on a naturally occurring (i.e. endogenously present) compound present in cells, e.g. a metabolite.

The term “hyperpolarization” means enhancing the nuclear polarization of the at least one NMR active nucleus in the marker. Upon enhancing the nuclear polarization of the at least one NMR active nucleus, the population difference between excited and ground nuclear spin states of the nucleus is significantly increased and thereby the MR signal intensity is amplified. The “hyperpolarization” of the at least one NMR active nucleus in the marker can be measured by its enhancement factor compared to thermal equilibrium at the spectrometer field and temperature. The term “hyperpolarized” denotes a nuclear hyperpolarization level in excess of 0.1%, more preferred in excess of 1%, even more preferred in excess of 10%, most preferred in excess of 30%.

The term “obtaining an NMR spectrum and/or an MR image” as used herein means that the tissue sample comprising the at least one hyperpolarized marker is subjected to an MR scanner, wherein the spectrum and/or image is provided as result of the scan.

The term “comparing” is used as common in the art and described below in more detail.

“Acceptable salts” or “salts” of the hyperpolarized marker may be metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like.

A “parameter” as used herein refers to a parameter, which can be deduced and/or calculated from the information obtained in the NMR spectrum and/or MR image. Preferably, such a parameter corresponds to a concentration of a specific substance, the amount of a specific substance, the spatial distribution of a specific substance, the accumulation of a specific substance, the influx rate of a specific substance, the pH of a sample, the increase or decrease in the amount or concentration of a specific substance over time, and the like.

A “reference” as used herein can refer to an NMR spectrum and/or MR image obtained under substantially identical conditions as used in the analysis of the potentially cancerous tissue sample, wherein said reference spectrum and/or image has been obtained in a tissue sample derived from healthy tissue of the identical tissue type. Further, the reference may be a predetermined reference (i.e. the reference analysis is not carried out in parallel to the method of the present invention) based on previous analysis of at least one reference tissue, preferably of at least 10 reference tissues, more preferably of at least 100 reference tissues and most preferably of more than 500 reference tissues. If the goal of the method resides e.g. in the diagnosis of breast cancer metastasis in sentinel lymph nodes, the reference tissue corresponds to at least one healthy sentinel lymph node. A “reference” may also be a specific threshold value to be compared to a corresponding value obtained from the spectrum and/or the image of the tissue sample.

The term “reference” also refers to a reference parameter with the above definition of a parameter. Such a reference parameter may have been obtained via an NMR spectrum and/or MR image in at least one healthy tissue sample (preferably at least about 10, more preferably at least about 50, even more preferably at least about 100, and most preferably at least more than 1000 healthy tissue samples); alternatively, said reference parameter may have been determined by other experimental means or may be derived from database information on healthy tissue/cells. Of course, the reference parameters also correspond to parameters such as a concentration of a specific substance, the amount of a specific substance, the spatial distribution of a specific substance, the accumulation of a specific substance, the influx rate of a specific substance, the pH of a sample, the increase or decrease in the amount or concentration of a specific substance over time, and the like, but determined in or known for healthy tissue or at least one healthy cell, preferably a healthy tissue sample comprised of viable cells.

The term “non-invasive” as used herein means that the tissue sample remains substantially intact; thus, the tissue sample may e.g. be used for further post-operative histopathology.

The terms “NMR” and “MR” as used herein refer to methods based on nuclear magnetic resonance and can either refer to spectroscopic or imaging investigations.

The term “T₁” is used as common in the field and refers to the longitudinal relaxation time constant. Thus, it corresponds to the decay constant for the recovery of the z component of the nuclear spin magnetization, M_{z}, towards its thermal equilibrium value.

The term “metabolic marker indicating metabolically active cells” as used herein refers to a marker, which is metabolized by a cell independent of the type of cell, i.e. whether the cell is a healthy cell or a malignant cell, and which results in a basically identical NMR spectrum and/or MR image of its metabolic products when normalized to a specific volume packed with either intact healthy or malignant cells, e.g. a voxel of 2×2×2 mm. “Basically identical” means in the above definition that the spectrum and/or image of healthy cells vs. malignant cells does preferably not deviate by a factor of >5, preferably of >3, and more preferably of >2. Clearly, the skilled person is able to define further markers within this definition, e.g. by carrying out experiments as shown in example 3.11 of the present application. Typically, the skilled person will start by identifying an identical metabolic pathway of the lymphocytes and the cancer cells (e.g. aerobic glycolysis) and then test metabolites comprised in this pathway (in the above example e.g. glucose). A further criterion for such a marker is that the different types of cells are able to metabolize the marker to a rather high degree, i.e. the metabolic products of said marker are detectable with a rather high signal intensity independent of the type of cells.

The term “metabolic marker allowing a distinction between lymphocytes and cancer cells” is also referred to as “metabolic contrast marker” in the present application and refers to a marker, which results in a substantially different NMR spectrum and/or MR image of its metabolic products when normalized to a specific volume packed with either intact healthy or malignant cells, e.g. a voxel of 2×2×2 mm. This difference may be based on a different metabolism of the metabolic contrast marker in healthy cells vs. malignant cells. “Substantially different” in the above definition means that the spectrum and/or image of healthy cells vs. malignant cells deviates by a factor of >5, preferably of >10, more preferably of >20 and most preferably of >30. Clearly, the skilled person is able to define further markers within this definition, e.g. by carrying out experiments as shown in examples 3.10 and 3.14 of the present application.

The term “metabolic product” refers to any product of the metabolic marker used, which can be detected due to its label in an NMR spectrum or MR image. For e.g. the metabolic marker ethyl acetoacetate, the metabolic product is acetoacetate; for e.g. the metabolic marker pyruvate, the metabolic product is lactate.

The term “exhibiting either a higher conversion/exhibiting a lower conversion” means that the metabolic products of the metabolic marker used are detected in higher/lower amounts and/or at a faster/slower conversion rate in the first type of cells as indicated compared to the second type of cells as indicated. The conversion is either normalized by cell number or by a given volume of cells (such as e.g. a voxel of 2×2×2 mm), wherein the cell number of a specific type of cells in such a volume depends on the cell size of the specific type of cells.

The terms “carboxylate ester” and “ester” are used herein interchangeably and as common in the field, i.e. as relating to a molecule comprising the following group: R₁—C(═O)—O—R₂, wherein one may refer to an “acid part” R₁—C(═O)—OH and an “alcohol part” HO—R₂. The term “short” as used in connection with a “short” alkyl-chain in the alcohol position comprised in an ester of low molecular weight refers to optionally branched carbon units ranging from 1 to 10, preferably of 1 to 9, more preferably from 1 to 8 and most preferably from 1 to 6 and to a benzyl moiety. The term “short” as used in connection with a “short” acid part comprised in an ester of low molecular weight refers to optionally branched carbon units ranging from 1 to 10, preferably of 1 to 9, more preferably from 1 to 8 and most preferably from 1 to 5.

The term “stable ester” means that the ester as used herein does substantially not hydrolyze under the conditions used for the claimed method, i.e. the conditions used for the hyperpolarization and the conditions used for contacting said marker with the sample and obtaining an NMR spectrum and/or a MR image.

The term “soluble ester” means that the ester as used herein is soluble or miscible in aqueous solution under the conditions used for the claimed method, i.e. the conditions used for the hyperpolarization and the conditions used for contacting said marker with the sample and obtaining an NMR spectrum and/or a MR image.

The term “ester of low molecular weight” refers to an ester of a molecular weight of up to 400 Da.

The term “functionalized” as used herein in connection with a straight or branched alkyl chain and/or aromatic group means that the chain and group, respectively, is substituted with a substituent selected from the group consisting of hydroxyl, oxo, halogen, amide, ketone and aldehyde.

The term “aromatic ester” refers to an ester comprising an aromatic group.

Further definitions are given in the next section on the method of the present invention.

### 2. Detailed Description of the Method of the Present Invention

In the following, the method according to the present invention will be outlined in further detail.

### The Obtained Tissue Sample

The surgical step of obtaining the tissue sample to be analyzed according to the method of the present invention is not part of the invention.

The obtained tissue sample potentially corresponds to cancer tissue, i.e. potentially comprises malignant cells. The tissue sample may be a tissue sample obtained from any part of the human or animal body. In preferred embodiments, the tissue sample corresponds to a lymph node or a sentinel lymph node. Further, the tissue sample may correspond to tissue surrounding the area, from which a (primary) tumor has been removed by surgery. Thus, the tissue sample may correspond to a rim of breast tissue remaining after removal of a breast tumor, or to a rim of prostate tissue after removal of a prostate tumor, or to a rim of colon tissue after removal of a colon tumor. The obtained tissue sample may typically have a size of 0.2 cm to 1 cm in diameter.

### Hyperpolarization of the Marker

In parallel to the surgery or alternatively prior to the surgery, the at least one marker according to the present invention is hyperpolarized. In any case, a timing for said hyperpolarization is used, which results in the at least one hyperpolarized marker being ready-for-use at the latest when the tissue sample has been obtained. The effect of hyperpolarization is present in a hyperpolarized marker, at a level that is useful for the present invention, for up to 3 times the T₁ value (3*T₁) of the hyperpolarized nucleus. This corresponds to between about 5 s to about 15 minutes, depending on the marker and type of hyperpolarized nucleus. Markers comprising nuclei with short T₁ (e.g. ¹H, ¹⁹F and ³¹P) have T₁'s up to about 10 s to about 15 s, markers comprising nuclei with medium T₁ (e.g. ¹³C) have T₁'s up to about 50 s to about 70 s and markers comprising nuclei with long T₁ (e.g. ¹⁵N) have T₁'s up to about 350 s.

To allow for equilibration reactions and/or uptake in cells and/or metabolic reactions, markers should preferably be contacted with the tissue sample within the elapse of two times the T₁ (2*T₁) of the hyperpolarized nucleus being ready-for-use. More preferably, the markers should be contacted with the tissue sample within the elapse of one time the T₁ (1*T₁) of the hyperpolarized nucleus being ready-for-use. Most preferably, the markers should be contacted with the tissue sample within the elapse of a half time the T₁ (0.5*T₁) of the hyperpolarized nucleus being ready-for-use.

Possible ways of hyperpolarization are known from the prior art and will be described only briefly in the following. In general, every way of hyperpolarizing a marker may be used. Usually, the tissue sample is contacted with a sample of 5 nmol to 250 mmol of hyperpolarized marker, preferably of 10 nmol to 125 mmol of hyperpolarized marker, more preferably 100 nmol to 60 mmol of hyperpolarized marker. A sample of 0.5 μmol to 5.0 μmol of the hyperpolarized marker is particularly preferred. The skilled person is aware that the amount of hyperpolarized marker used will inter alia depend on the contacting step used; thus, if the tissue sample is contacted in step a) with said at least one hyperpolarized marker by injection, a lower amount of said marker may be sufficient if compared to a contacting step by perfusion, wherein a larger amount of said marker might be needed.

One way of hyperpolarizing a marker resides therein that polarization is imparted to the NMR active nuclei in the marker by thermodynamic equilibration at a very low temperature and high field. Hyperpolarization compared to the operating field and temperature of the NMR magnet is affected by use of a very high field and very low temperature (brute force). The magnetic field strength used should be as high as possible. Suitably, the magnetic field strength is higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another way of hyperpolarizing a marker is the parahydrogen method. An unsaturated chemical or biological precursor of the marker comprising hydrogenatable carbon-carbon double- or triple bonds is exposed to parahydrogen-enriched hydrogen gas in the presence of a suitable catalyst. The enriched hydrogen will then react with the precursor imparting a non-thermodynamic spin configuration to the target molecule. The parahydrogen method is described e.g. in WO 99/24080 or WO 00/71166, both of which are incorporated herein by reference.

In an alternative parahydrogen method, the “Signal Amplification By Reversible Exchange (SABRE)”, the marker is hyperpolarized after contacting with a metal dihydride, derived from parahydrogen enriched hydrogen gas. In the SABRE method no chemical change is introduced to the molecule upon hyperpolarization and so no precursor compound for the marker is needed (Ducket et al. Science 2009, vol 323, 5922, 1708-1711).

A preferred way of hyperpolarizing the marker is the DNP (dynamic nuclear polarization) method effected by a polarizing agent or a so-called DNP agent, a compound comprising unpaired electrons. DNP mechanisms include the Overhauser effect, the solid effect and the thermal mixing effect. Many known paramagnetic compounds may be used as DNP agents, e.g. transition metals such as chromium (V) ions, organic free radicals such as nitroxide radicals, BDPA and trityl radicals (see e.g. WO 98/58272) or other molecules having associated free electrons. During the DNP process, energy, normally in the form of microwave radiation, is provided, which will initially excite the paramagnetic species. Upon decay to the ground state, there is a transfer of polarization to the NMR active nuclei of the marker. The method may utilize a moderate or high magnetic field and very low temperature, e.g. by carrying out the DNP process in liquid helium and a magnetic field of about 0.5 T or above. Alternatively, a moderate magnetic field and any temperature at which sufficient NMR enhancement is achieved may be employed. The DNP technique is e.g. further described in WO 98/58272 and WO 01/96895, both of which are included herein by reference. The method may be carried out by using a first magnet for providing the polarizing magnetic field and a second magnet for providing the primary field for MR spectroscopy. Alternatively, both DNP polarization and NMR spectroscopy may be carried out in a single magnet.

To polarize a marker to be used in the method of the present invention by the DNP method, a composition of the compound to be polarized and a DNP agent is prepared which is then optionally frozen and inserted into a DNP polarizer (in which the compound composition will freeze at the low temperature if it has not been frozen before) for polarization. After the polarization, the frozen solid hyperpolarized composition is rapidly transferred into the liquid state either by melting it or by dissolving it in a suitable dissolution medium. Suitable devices for the dissolution and melting process are e.g. described in WO 02/37132 and in WO 02/36005, both of which are incorporated herein by reference.

In order to obtain a high polarization level on the marker to be polarized, said marker and the DNP agent need to be evenly distributed for the DNP process to be effective. This is not the case if the composition crystallizes upon being frozen or cooled. If the marker or a chemical precursor to the marker do not form an amorphous structure in the composition (form a “glass”) then a so-called glass former may be added to the composition to prevent crystallization of the solid composition. Examples of preferred “glass-formers” in the contact of the invention are compounds such as di- or polyols, e.g. ethylene glycol or glycerol, crown ethers or DMSO. Since the present method is carried out in vitro, any type of glass-formers may be used.

The DNP agent plays a decisive role in the DNP process as its choice has a major impact on the level and polarization build-up time, which can be achieved on and for the hyperpolarized marker. Suitable DNP agents are inter alia transition metals such as chromium(V) ions, magnetic particles or organic free radicals such as nitroxide radicals, BDPA radicals and trityl radicals (see also WO 99/35508, “OMRI contrast agents”). Thus, if a trityl radical is used as DNP agent, a suitable concentration of such a trityl or BDPA radical is 1-100 mM, preferably 5-50 mM, more preferably 8-30 mM in the composition used for DNP. If a Nitroxyl radical is used as DNP agent, a suitable concentration of such a nitroxyl radical is 1-100 mM, preferably 10-80 mM, more preferably 20-50 mM in the composition used for DNP.

The composition undergoing DNP may further comprise a paramagnetic metal ion. It has been found that the presence of paramagnetic metal ions may result in increased polarization levels in the marker to be polarized by DNP, as e.g. described in WO 2007/064226, which is incorporated herein by reference. If a paramagnetic metal ion is added to the composition, a suitable concentration of such a paramagnetic metal ion is 0.1-6 mM (metal ion) in the composition, and a concentration of 0.5-3 mM is preferred.

Preferably, the DNP method is carried out as briefly described in the following steps:
(a) contacting the marker with a polarizing agent (DNP preparation) and optionally with a glass-forming agent and optionally with a chelate of a paramagnetic metal ion;
(b) Placing the DNP preparation in the presence of a magnetic field and low temperature; and exposing said polarizing agent to a microwave irradiation of a frequency selected to excite electron spin transitions in said polarizing agent;
(c) Dissolving said DNP preparation in an aqueous carrier (hyperpolarized solution).
   An efficient DNP process is best obtained at high magnetic field (0.5-10 T) and low temperatures (0.5-10 K).

If the hyperpolarization is carried out by a method that requires the marker sample to be in the solid state (such as the preferred DNP-method), the marker sample is preferably brought into solution after the hyperpolarization step. Suitable solvents for this step are inter alia selected from buffers with a concentration range of 5 to 100 mM (40 mM being particularly preferred) and a pH preferably at about physiological pH (and thus around pH 7.3), wherein it is preferred to use the following buffers: MES, citrate, maleate, bis-TRIS, phosphate, bicarbonate, MOPS, HEPES, TEA. Preferably, the volume after dissolution is within a range of between 100 μl and 10 ml, more preferably 200 μl to 5 ml, most preferably between 500 μl and 2 ml.

Thus, summarizing the above section on hyperpolarization, a hyperpolarized marker dissolved in a buffer is prepared and provided prior to carrying out step a) of the present method. For example, 0.05 mmol to 0.5 mmol of the hyperpolarized marker prepared by the DNP-method may be dissolved in 5 ml phosphate buffer, 40 mM, pH 7.3.

### Step a) of the Method

In this step, the obtained tissue sample is contacted with the at least one hyperpolarized marker. This contacting step may e.g. be carried out by transferring the solution of the at least one hyperpolarized marker described above into a syringe attached to a fine needle. A volume of between 10 μl to 500 μl may then be injected into the tissue sample. The volume inter alia depends on the starting amount of the marker, the degree of hyperpolarization, and the dissolution volume and can easily be determined by the skilled person. In the above example of 0.05 mmol to 0.5 mmol of hyperpolarized marker prepared by the DNP-method and dissolved in 5 ml phosphate buffer, 40 mM, pH 7.3, one may e.g. inject between 10 μl to 500 μl, preferably 25 μl to 200 μl, such as e.g. 25 μl, 50 μl, 100 μl, 150 μl or 200 μl. An alternative administration resides in the perfusion of the marker into the tissue sample, in which case the volume of hyperpolarized marker solution may be higher e.g. 0.5 ml to 5 ml, preferably 1 ml to 3 ml. This may be carried out within a magnetic field.

Step a) may also be carried out more than once in the method of the present invention; it may be repeated and e.g. be carried out twice or three times during the method of the present invention.

Step a) may also be carried out where several tissues are analyzed sequentially or at the same time and where e.g. several lymph nodes have been placed in a double, triple or more chambered vessel.

Prior to or subsequent to step a), the tissue sample (which is preferably present in a vessel, more preferably in a vessel with buffer) is placed in a MR scanner which has been tuned to the nucleus of interest (i.e. in accordance with the hyperpolarized marker used) and shimmed on a phantom resembling the tissue sample.

### Step b) of the Method

An NMR spectrum and/or an MR image of the tissue sample comprising the hyperpolarized marker is then obtained according to standard procedures. This of course also includes the detection of metabolic products of the at least one metabolic marker. Said step is carried out following an incubation period after completion of step a), wherein said incubation period is dependent on the nucleus of the hyperpolarized marker. Generally, such an incubation period may be between 1 second and 10 minutes. More specifically, the incubation period is preferably from about 1 to about 5 minutes, more preferably 1 minute or 2 minutes or 3 minutes for ¹⁵N. The incubation period is preferably about 5 seconds to 60 seconds, more preferably 10 seconds or 20 seconds or 30 seconds or 40 seconds for ¹³C. In a preferred embodiment, the incubation period corresponding to the incubation time of the hyperpolarized marker in the tissue sample is standardized.

The NMR spectrum generated may be a one-, two- or multidimensional NMR spectrum, preferably a one-dimensional NMR spectrum of the nucleus of choice, like ¹³C, ¹⁵N, ¹⁹F, ³¹P or ¹H in accordance with the hyperpolarized marker used. The spectrum may be acquired in a single scan or in several scans with any combination of RF and gradient pulses. Obtained values can e.g. comprise a chemical shift, line broadening, and dipolar or scalar couplings. In a preferred embodiment, low flip angels are used in the generation of the NMR spectrum. It may thus be possible to study the time dependent fate of the marker. In another preferred embodiment, the NMR analysis is an image (e.g. a CSI) providing spatial information of the marker and/or metabolites thereof.

### Determined Signals and Parameters

Depending on the type of marker used and/or the parameter to be determined from said obtained NMR spectrum and/or MR image, it can be useful to determine the signal(s) of at least one metabolite of said marker, optionally in addition to the signal of the at least one marker. However, the obtained NMR spectrum itself and/or the obtained MR image itself may already provide sufficient information in order to carry out step d) as described below; in this respect, the above mentioned values such as e.g. a chemical shift, line broadening, dipolar or scalar couplings may be used and it should be noted that such NMR spectrum and/or MR image information may in general be used in steps c) and d) as described below with all kinds of markers including the ones described in further detail in the following.

If a marker taken up by the cells is used in the method according to the present invention, it is preferred to determine and quantify the signal of said at least one marker in the tissue sample (see also examples 1 and 2 below). One may then further calculate a specific concentration of said marker in the tissue sample as parameter. If an MR image should have been obtained, one may also calculate a specific spatial distribution of said marker in the tissue sample as parameter; in doing so, e.g. an accumulation in a certain area may be visualized.

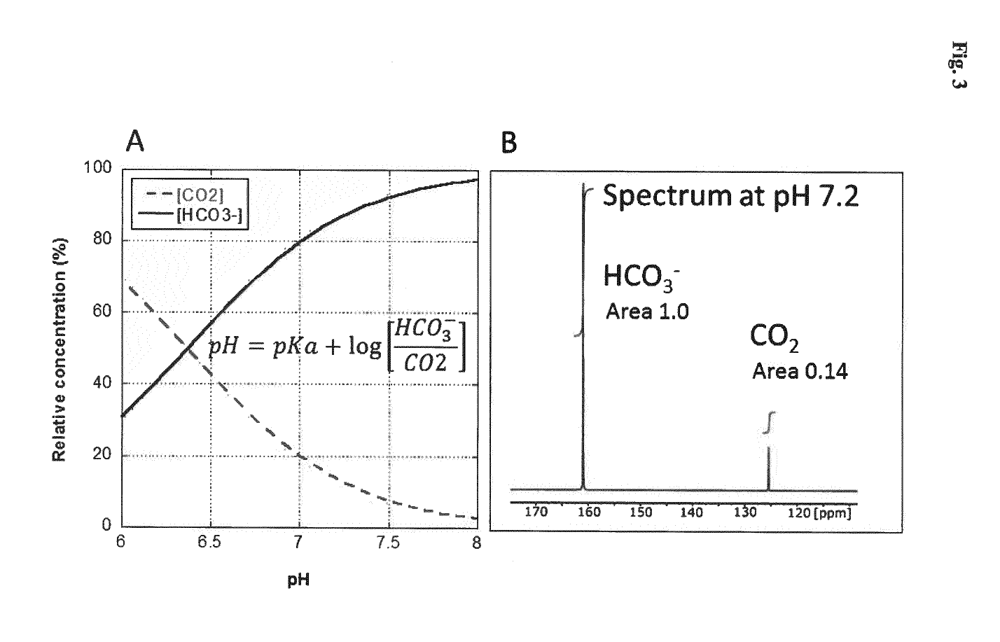

If a pH sensitive marker is used in the method according to the present invention, it is preferred to determine and quantify the signals of said marker (e.g. ¹³C bicarbonate) and a pH-dependent metabolite of said marker (e.g. ¹³C carbon dioxide, see also example 3 below). Using the quantified signals of these two molecules, the concentrations of the two molecules can be determined; using the concentrations, the pH value in the tissue sample as parameter may then be determined according to the Henderson-Hasselbalch equation (see also FIG. 3). It can also be preferred to determine and quantify the chemical shift of said marker as a mean to determine the extracellular pH, e.g. using phosphonate compounds such as 2-amino-phosphono-carboxylic acids with pKas in the physiological range, fluorinated alpha amino acids such as difluoromethylalanine with a pKa of 7.3 resulting in two signals separated with a chemical shift and directly indicative of the extracellular pH. In another preferred embodiment related to the intracellular pH, ester variants of the just-mentioned compounds are used for the measurement of the intracellular pH. As above, a spatial distribution of these molecules as parameter could also be determined.

If a metabolic marker is used in the method according to the present invention, it is preferred to determine and quantify the signal of at least one metabolite of said marker. Examples in this respect are 1-¹³C pyruvate as marker and 1-¹³C lactate or 1-¹³C alanine as metabolites, or 1,4-¹³C₂ fumaric acid as marker and 1,4-¹³C₂ malate as metabolite, or 1-¹³C ketoisocaproic acid as marker and 1-¹³C leucine as metabolite, or 5-¹³C glutamine acid as marker and 5-¹³C glutamate as metabolite (see also examples 4 to 9 below). One may then calculate a specific concentration of the at least one metabolite in the tissue sample as parameter. One may also determine a ratio of the marker and the metabolite. If an MR image should have been obtained, one may also calculate a specific spatial distribution of the at least one metabolite in the tissue sample as parameter; in doing so, e.g. an accumulation in a certain area may be visualized.

The quantification of signals and the determination of specific parameters such as the parameters discussed above correspond to routine tasks for the skilled person; corresponding analysis software is commonly used with MR devices.

### Step c) of the Method

In step c), the NMR spectrum and/or the MR image obtained in step b) and/or at least one parameter determined from said NMR spectrum and/or said MR image is compared to a reference as defined above. Alternatively, step c) refers to the determination of a specific ratio r_{S} of the metabolic products of two metabolic markers used; such a determination is clearly routine for the skilled person and thus not further discussed herein.

As discussed above, the obtained NMR spectrum itself and/or the obtained MR image itself may already provide enough information to carry out the assignment step d). Thus, in one embodiment, the obtained spectrum and/or image is compared to a reference NMR spectrum and/or a reference MR image obtained from at least one healthy tissue sample as defined above.

Secondly, a parameter which can be determined from the NMR spectrum and/or the MR image can be compared to a reference parameter as defined above.

The comparison-step according to the present method may be an automated comparison by software, which is preferably used with MR devices. Of course, the reference spectral information itself or the specific parameters of references are preferably already included in the analysis software.

### Step d) of the Method

In step d), the presence or absence of cancer in said tissue sample is then determined, i.e. cancer is assigned to the tissue sample based on the above comparison.

Clearly, if there is no substantial difference between the NMR spectrum and/or the MR image of the tissue sample and/or the at least one parameter determined from said obtained NMR spectrum and/or MR image when compared to the reference, the analyzed tissue sample fails to comprise malignant cells and no cancer is assigned to said tissue sample.

However, if there is a substantial difference between the NMR spectrum and/or the MR image of the tissue sample and/or the at least one parameter determined from said obtained NMR spectrum and/or MR image when compared to the reference, the analyzed tissue sample corresponds to cancer tissue, e.g. a metastasis, and cancer is accordingly assigned to the tissue sample. With respect to the above mentioned parameters, an increased uptake, a more acidic pH value and an increased metabolism may be indicative of cancer. However, this depends on the hyperpolarized marker used and the type of cancer.

Alternatively, step d) refers to a comparison of ratio r_{S} to a reference ratio r_{R}, which can easily be carried out in an automated manner as described above.

### Step e) of the Method

This step refers to the assignment of cancer to a lymph node tissue sample, wherein said assignment is based on the comparison carried out in the previous step.

Preferably, steps b) to d) and b) to e), respectively, are carried out in an automated manner. This means that references as described above have been gained and validated and that the step of obtaining an NMR spectrum and/or an MR image, the step of comparing to a reference, and the step of assigning cancer are carried out by an integrated MR-analysis device.

### Advantages of the Present Method

It is immediately apparent from the above that the claimed method corresponds to a fast method; thus, it may be carried out within about 5 to about 20 minutes. Clearly, a cancer surgery is still ongoing within such a time frame such that the present method is referred to as “intra-operatively” (see also above).

Secondly, it is apparent that the present method allows for a reliable result; the present method is very sensitive but yet also very specific due to the use of at least one hyperpolarized marker in combination with MR detection.

Further, the steps as carried out in the present method do not result in a disruption of the tissue sample; subsequent to the present method, the tissue may still be used for further purposes such as e.g. a histopathology; Thus, the present method may also be referred to as “non-invasive” method (see also above).

### Further Optional Steps of the Method

Further optional steps of the present method have already been described in the above objects and summary section. As can be derived therefrom, one may e.g. introduce a further step prior to the contacting of the sample with the hyperpolarized marker; all substances discussed in detail above may result in an increased specificity and sensitivity of the method. Further, since the present method corresponds to an in vitro method, there are no limitations with respect to such substances which would e.g. apply in vivo.

Finally, it is also possible to introduce an additional step after step a) and prior to step b), wherein a paramagnetic relaxation agent is added to the tissue sample. Particularly in the setup of a marker taken up by cells, such an additional step is clearly suited to increase the detection sensitivity and specificity since no extracellular marker is detected any more.

Particularly preferred embodiments of the present invention relate to:
1. Method of intra-operatively diagnosing cancer in a tissue sample, wherein said method is carried out on an excised tissue sample obtained from a patient suffering from cancer and wherein said method comprises the following steps:
   a) Contacting said tissue sample with at least one hyperpolarized marker;
   b) Obtaining an NMR spectrum and/or an MR image of said tissue sample;
   c) Comparing said NMR spectrum and/or said MR image obtained in step b) and/or at least one parameter determined from said obtained NMR spectrum and/or MR image to a reference, wherein said reference corresponds to an NMR spectrum and/or an MR image of a healthy tissue sample and/or at least one parameter of a healthy tissue sample; and
   d) Assigning cancer to said tissue sample based on the comparison carried out in step c), wherein a difference in the NMR spectrum and/or MR image and/or at least one parameter of said tissue sample and said reference is indicative of cancer.
2. Method according to 1, wherein said steps are not conducted on the human body.
3. Method according to 1 or 2, wherein said at least one hyperpolarized marker contains at least one NMR active nucleus and is selected from the group consisting of acetate, acetoacetate, alanine, 2-oxoglutarate, arginine, asparagine, aspartate, beta-alanine, trimethylglycine, bicarbonate, butyrate, choline, cis-aconitic acid, creatine, cysteate, cysteine, fructose, fumarate, glucose, glutamate, glutamine, glycine, glyoxylic acid, guanidinoacetic acid, homocysteine, 4-hydroxyproline, 3-hydroxybutyrate, hydroxypyruvate, 2-ketoisocaproic acid, lactic acid, malic acid, methionine, N-acetyl aspartate, N-acetyl cysteine, oxaloacetate, phenylalanine, phenylpyruvate, proline, pyruvate, serine, taurine, ureidopropionate, isotopically enriched compounds thereof, salts thereof, esters thereof and mixtures thereof, wherein said marker preferably contains and/or is preferably enriched with ¹³C and/or ¹⁵N.
4. Method according to any one of 1 to 3, wherein said tissue sample is contacted in step a) with said at least one hyperpolarized marker by injection and/or perfusion.
5. Method according to any one of 1 to 4, wherein said cancer is selected from breast cancer, prostate cancer and colon cancer, preferably breast cancer.
6. Method according to any one of 1 to 5, wherein said tissue sample is a lymph node tissue sample, preferably a sentinel lymph node tissue sample.
7. Method according to any one of 1 to 6, wherein a cancer metastasis is diagnosed in said tissue sample.
8. Method according to any one of 1 to 7, wherein said hyperpolarized marker is a marker taken up by cells and said difference in the NMR spectrum and/or MR image and/or said at least one parameter of said tissue sample and the reference is caused by an increased or decreased uptake of said marker by cancer cells compared to healthy cells, preferably an increased uptake.
9. Method according to 8, wherein said marker taken up by cells is selected from the group consisting of ¹⁵N choline, ¹⁵N trimethylglycine, 1-¹³C acetate, 1-¹³C butyrate, 1-¹³C beta-alanine and 1- or 2-¹³C,²H₂ taurine, salts thereof, esters thereof and mixtures thereof
10. Method according to any one of 1 to 7, wherein said hyperpolarized marker is a pH sensitive marker and said difference in the NMR spectrum and/or MR image and/or said at least one parameter of said tissue sample and the reference is caused by a pH increase or decrease in cancer tissue compared to healthy tissue, preferably a pH decrease.
11. Method according to 10, wherein said pH sensitive marker is selected from the group consisting of ¹³C bicarbonate, ¹⁹F difluoromethylalanine, salts thereof, esters thereof and mixtures thereof
12. Method according to any one of 1 to 7, wherein said hyperpolarized marker is a metabolic marker and said difference in the NMR spectrum and/or MR image and/or said at least one parameter of said tissue sample and the reference is caused by an increased or decreased metabolic profile in cancer tissue compared to healthy tissue, preferably an increased metabolic profile.
13. Method according to 12, wherein said metabolic marker contains at least one NMR active nucleus and is selected from the group consisting of acetoacetate, 2-oxoglutarate, aspartate, fumarate, glucose, glutamine, 3-hydroxybutyrate, 2-ketoisocaproic acid, pyruvate, isotopically enriched compounds thereof, salts thereof, esters thereof and mixtures thereof, wherein said marker preferably contains and/or is preferably enriched with ¹³C and/or ¹⁵N.
14. Method according to any one of 1 to 13, wherein said method comprises at least one further step preceding step a), namely
   Removing non-adherent cells from said tissue sample; and/or
   Transferring said tissue sample to a vessel; and/or
   Contacting said tissue sample with a buffer, preferably a physiological buffer; and/or
   Contacting said tissue sample with at least one substance selected from the group consisting of an unlabeled co-substrate; an inhibitor specifically blocking the uptake of specific substances into healthy cells; a substance decreasing or increasing the pH; and a substance specifically increasing enzymatic activity in cancer cells compared to healthy cells; and/or
   Tempering said tissue sample;
and/or wherein said method comprises at least one further step between step a) and step b), namely
   Contacting said tissue sample with at least one paramagnetic relaxation agent resulting in the loss of hyperpolarization of said at least one hyperpolarized marker in the extracellular compartment.
15. Method according to any one of 1 to 14, wherein said hyperpolarized marker is hyperpolarized by dynamic nuclear polarization (DNP).

### 3. EXAMPLES

### 3.1. Example 1

### Detecting Cancer Metastases in Sentinel Lymph Nodes Through Differentiated Uptake of ¹⁵N Choline

Step 1: a sample of ¹⁵N choline, 0.05 mmol, is hyperpolarized according to procedures known in the art (Allouche-Arnon et al. Contrast Media Mol Imaging. 2011 November-December; 6(6):499-506).

Step 2: a sentinel lymph node from a prostate cancer patient undergoing prostate cancer surgery for a primary tumor in the prostate is identified, excised and rapidly (preferably within one minute from completed excision) placed in a 4 ml vessel filled with Ringer's solution tempered to 37° C.

Step 3: the vessel containing the lymph node is placed in an MR scanner which has been tuned to ¹⁵N and shimmed on a phantom resembling the vessel with the lymph node.

Step 4: the hyperpolarized marker is dissolved in 5 ml phosphate buffer (40 mM, pH 7.3) and otherwise prepared for use according to procedures known in the art (Ardenkjaer-Larsen et al. Proc Natl Acad Sci USA. 2003 Sep. 2; 100(18):10158-63). The solution containing the hyperpolarized marker is transferred to a syringe.

Step 5: a fraction of the solution, 4 ml, containing the hyperpolarized marker is perfused into the lymph node.

Step 6: 120 s after perfusion of the solution containing the hyperpolarized marker, Omniscan, 200 μl, is injected into the lymph node to equilibrate the extra-cellular marker to thermal Bolzmann distribution.

Step 7: 10 s after injection of Omniscan, a ¹⁵N MR spectroscopic investigation in performed on the lymph node, the signal from ¹⁵N choline is quantified and compared to a standard value obtained from a healthy lymph node. An increased uptake in the tissue sample as determined by higher signals in the ¹⁵N MR spectrum or by a higher concentration of choline indicates the presence of cancer cells in the lymph node. The operating surgeon is notified about the outcome of the investigation.

Step 8: the lymph node is removed from the Ringer's buffer and prepared for histology.

### 3.2. Example 2

### Detecting Cancer Metastases in Sentinel Lymph Nodes Through Differentiated Uptake of 1-¹³C Acetate

Step 1: a sample of 1-¹³C acetate, 0.05 mmol, is hyperpolarized according to procedures known in the art (Jensen et al. J Biol Chem. 2009 Dec. 25; 284(52):36077-82).

Step 2: a sentinel lymph node from a breast cancer patient undergoing breast cancer surgery for a primary tumor in the breast is identified, excised and rapidly (preferably within one minute from completed excision) placed in a 2 ml vessel filled with Ringer's solution tempered to 37° C.

Step 3: the vessel containing the lymph node is placed in an MR scanner which has been tuned to ¹³C and shimmed on a phantom resembling the vessel with the lymph node.

Step 4: the hyperpolarized marker is dissolved in 5 ml phosphate buffer (40 mM, pH 7.3) and otherwise prepared for use according to procedures known in the art (Ardenkjaer-Larsen et al. Proc Natl Acad Sci USA. 2003 Sep. 2; 100(18):10158-63). The solution containing the hyperpolarized marker is transferred to a syringe with a fine needle.

Step 5: a fraction of the solution, 50 μl, containing the hyperpolarized marker is injected into the lymph node.

Step 6: 15 s after injection of the solution containing the hyperpolarized marker, Omniscan, 100 μl, is injected into the lymph node to equilibrate the extra-cellular marker to thermal Bolzmann distribution.

Step 7: 5 s after injection of Omniscan, a ¹³C MR spectroscopic investigation in performed on the lymph node, the signal from ¹³C acetate is quantified and compared to a standard value obtained from a healthy lymph node. An increased uptake in the tissue sample as determined e.g. by higher signals in the ¹³C MR spectrum or by a higher concentration of acetate indicates the presence of cancer cells in the lymph node. The operating surgeon is notified about the outcome of the investigation.

Step 8: the lymph node is removed from the Ringer's buffer and prepared for histology.

### 3.3. Example 3

### Detecting Cancer Metastases in Sentinel Lymph Nodes Through pH Differences

Step 1: a sample of ¹³C bicarbonate, 0.1 mmol, is hyperpolarized according to procedures known in the art (Gallagher et al. Nature. 2008 Jun. 12; 453(7197):940-3).

Step 2: a sentinel lymph node from a colon cancer patient undergoing colon cancer surgery for a primary tumor in the colon is identified, excised and rapidly (preferably within one minute from completed excision) placed in a 1 ml vessel filled with Ringer's solution tempered to 37° C.

Steps 3 to 4 are performed as in example 2.

Step 5: a fraction of the solution, 25 μl, containing the hyperpolarized marker is injected into the lymph node.

Step 6: after 10 to 30 s, a ¹³C MR spectroscopic investigation is performed on the lymph node and the signals from ¹³C bicarbonate and ¹³C carbon dioxide are quantified. The pH is calculated according to the Henderson-Hasselbalch equation (see FIG. 3) and compared to a standard value obtained from a healthy lymph node. A more acidic pH in the analyzed lymph node (i.e. <pH 7.2) indicates the presence of cancer cells. The operating surgeon is notified about the outcome of the investigation.

Step 7: the lymph node is removed from the Ringer's buffer and prepared for histology.

### 3.4. Example 4

### Detecting Cancer Metastases in Sentinel Lymph Nodes Through Metabolic Differences

Step 1: a sample of 1,4-¹³C₂ fumaric acid, 0.05 mmol, is hyperpolarized according to procedures known in the art (Gallagher et al Proc Natl Acad Sci USA. 2009 Nov. 24; 106(47):19801-6).

Steps 2 to 4 are performed as in example 2.

Step 5: a fraction of the solution, 100 μl, containing the hyperpolarized marker is injected into the lymph node.

Step 6: after 30 s, a ¹³C MR spectroscopic investigation is performed on the lymph node and the signal from the metabolite 1,4-¹³C₂ malate is quantified and compared to a standard value obtained from a healthy lymph node. An increased metabolism in the analyzed lymph node as determined e.g. by a higher signal of at least one of the above metabolites in the ¹³C MR spectrum or by a higher concentration of at least one of the above metabolites indicates the presence of cancer cells in the lymph node. The operating surgeon is notified about the outcome of the investigation.

Step 7: the lymph node is removed from the Ringer's buffer and prepared for histology.

### 3.5. Example 5

### Detecting Cancer Metastases in Sentinel Lymph Nodes Through Metabolic Differences after Addition of a Co-Substrate

Step 1: a sample of 1-¹³C ketoisocaproic acid, 0.1 mmol, is hyperpolarized according to procedures known in the art (Karlsson et al. Int J Cancer. 2010 Aug. 1; 127(3):729-36).

Step 2: a sentinel lymph node from a prostate cancer patient is identified, excised and rapidly (preferably within one minute from completed excision) placed in a 2 ml vessel filled with Ringer's solution tempered to 37° C. containing the co-substrate glutamate in a range of from 5 mM up to 100 mM.

Steps 3 to 4 are performed as in example 2.

Step 5: a fraction of the solution, 50 μl, containing the hyperpolarized marker is injected into the lymph node.

Step 6: after 25 s, a ¹³C MR spectroscopic investigation is performed on the lymph node, the signal from the metabolite 1-¹³C leucine is quantified and compared to a standard value obtained from a healthy lymph node. An increased metabolism in the analyzed lymph node as determined e.g. by a higher signal of 1-¹³C leucine in the ¹³C MR spectrum or by a higher concentration of leucine indicates the presence of cancer cells in the lymph node. The operating surgeon is notified about the outcome of the investigation.

Step 7: the lymph node is removed from the Ringer's buffer and prepared for histology.

### 3.6. Example 6

### Detecting Cancer Metastases in Sentinel Lymph Nodes Through Metabolic Differences after Addition of an Activating Substance

Step 1: a sample of 5-¹³C glutamine, 0.2 mmol, is hyperpolarized according to procedures known in the art (Jensen et al. Chemistry. 2009 Oct. 5; 15(39):10010-2).

Step 2: a sentinel lymph node from a breast cancer patient is identified, excised and rapidly (preferably within one minute from completed excision) placed in a 4 ml vessel filled with 100 mM phosphate buffer tempered to 37° C. The buffer (comprising a high phosphate concentration not tolerated in vivo) enhances the activity of the glutaminase 1 enzyme, which is overexpressed in cancer cells compared to non-cancerous cells.

Steps 3 to 4 are performed as in example 2.

Step 5: a fraction of the solution, 2 ml, containing the hyperpolarized marker is perfused into the lymph node.

Step 6: after 10 s, a ¹³C MR spectroscopic investigation is performed on the lymph node, the signal from the metabolite 5-¹³C glutamate is quantified and compared to a standard value obtained from a healthy lymph node. An increased metabolism in the analyzed lymph node as determined e.g. by a higher signal of 5-¹³C glutamate in the ¹³C MR spectrum or by a higher concentration of glutamate indicates the presence of cancer cells in the lymph node. The operating surgeon is notified about the outcome of the investigation.

Step 7: the lymph node is removed from the Ringer's buffer and prepared for histology.

### 3.7. Example 7

### Detecting Cancer Metastases in Sentinel Lymph Nodes Through Metabolic Differences after Addition of a Transport Inhibitor

Step 1: a sample of 1-¹³C lactic acid, 0.1 mmol, is hyperpolarized according to procedures known in the art (WO 2009/013350).

Step 2: a sentinel lymph node from a prostate cancer patient is identified, excised and rapidly (preferably within one minute from completed excision) placed in a 1 ml vessel filled with Ringers solution tempered to 37° C.; p-Chloromercuribenzoic acid (pCMBS) is added to a final concentration of 1 mM and the sample is incubated for 5 minutes before proceeding with step 3. pCMBS inhibits the monocarboxylic transporters 1 and 4 (MCT1 and MCT4), which are active in both normal and cancerous cells, whereas it does not inhibit the MCT 2 which is only active in cancer cells.

Steps 3 to 4 are performed as in example 2.

Step 5: a fraction of the solution, 50 μl, containing the hyperpolarized marker is injected into the lymph node.

Step 6: after 15 s, a ¹³C MR spectroscopic investigation is performed on the lymph node, the signal from the metabolites 1-¹³C pyruvate and 1-¹³C alanine is quantified and compared to a standard value obtained from a healthy lymph node treated in a similar manner. An increased metabolism in the analyzed lymph node as determined e.g. by a higher signal of 1-¹³C pyruvate and/or 1-¹³C alanine in the ¹³C MR spectrum indicates the presence of cancer cells in the lymph node. The operating surgeon is notified about the outcome of the investigation.

Step 7: the lymph node is removed from the Ringer's buffer and prepared for histology.

### 3.8. Example 8

### In Cell ¹³C-NMR Detection of 1-¹³C-Lactate Using 1-¹³C-Pyruvate as Hyperpolarized Marker

Step 1: 1-¹³C pyruvic acid (1286 mg, 14.4 mmol) was mixed with 25 mg of trityl radical (Tris(8-carboxy-2,2,6,6-(tetra(methoxyethyl)-benzo-[1,2-4,5′]-bis-(1,3)-dithiole-4-yl)-methyl sodium salt) (15 mM) and gadolinium trimeric complex (Gadolinium chelate of 1,3,5-tris-(N-(DO3A-acetmido)-N-methyl-4-amino-2-methylphenyl)-[1,3,5]triazinane-2,4,6-trione)) (0.6 mM). A sample of this solution (9.2 mg) was hyperpolarized for 60 minutes, at 93.900 GHz and 1.3 K. The sample was dissolved in phosphate buffer (4 ml, pH 7.3, 40 mM) with addition of sodium hydroxide (11 μl, 12M) to neutralize the acid in the hyperpolarized sample. The hyperpolarized solution was further diluted with tempered (37° C.) buffer to 30 ml.

Step 2: 500 μl of hyperpolarized sodium 1-¹³C-pyruvate from step 1 was mixed into 5 million prostate cancer cells (PC-3, human prostate cell adenocarcinoma) in a 10 mm NMR tube and placed in a 9.4 T NMR magnet. Signals of 1-¹³C-pyruvate and 1-¹³C-lactate were detected by acquiring a set of ¹³C-MR spectra every 3 s with a 15 degree RF pulse and the signal from 1-¹³C-lactate was quantified.

Using an identical protocol as outlined above, a second study was performed in healthy prostate cells (PNT-1A, immortalized healthy prostate cells).

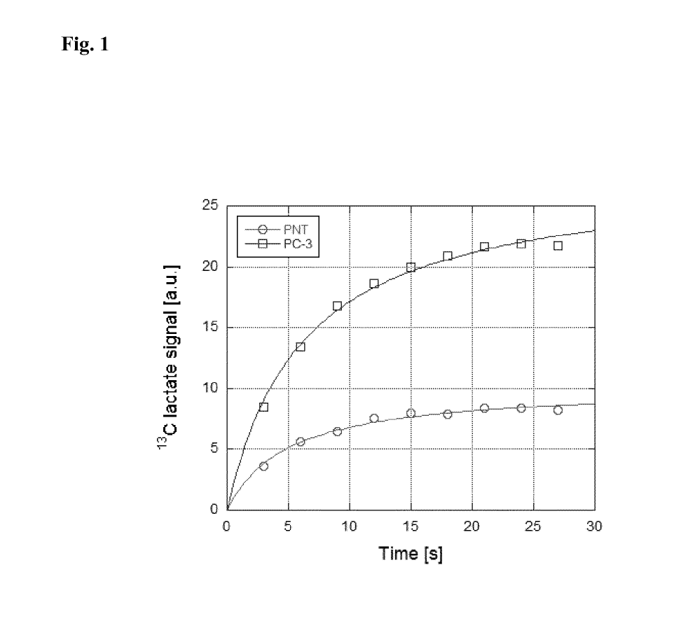

The results of the two experiments are compared in FIG. 1, and it can clearly be derived from the comparison that the 1-¹³C-lactate signal is increased in PC-3 cells compared to non-cancerous prostate cells indicating an increased metabolism in PC-3 cells.

### 3.9. Example 9

### In Cell ¹³C-NMR Detection of 1,4-¹³C₂-Malate Using 1,4-¹³C₂-Fumarate as Hyperpolarized Marker

Step 1: 1,4-¹³C₂ Fumaric acid (274 mg, 2.32 mmol) was dissolved in a DMSO solution (1660 μl) of trityl radical (Tris(8-carboxy-2,2,6,6-(tetra(methoxyethyl)-benzo-[1,2-4,5′]-bis-(1,3)-dithiole-4-yl)-methyl sodium salt) (19 mM) and gadolinium trimeric complex (Gadolinium chelate of 1,3,5-tris-(N-(DO3A-acetmido)-N-methyl-4-amino-2-methylphenyl)-[1,3,5]triazinane-2,4,6-trione)) (0.8 mM). A sample of this solution (32.5 mg) was hyperpolarized for 60 minutes, at 93.900 GHz and 1.3 K. The sample was dissolved in phosphate buffer (4 ml, pH 7.3, 40 mM) with addition of sodium hydroxide (22 μl, 12M) to neutralize the acid in the hyperpolarized sample.

Step 2: 100 μl of hyperpolarized sodium 1,4-¹³C₂-fumarate from step 1 was mixed into 500 μl RPMI medium containing 5 million breast cancer cells (MDA-MB-231, human epithelial breast cancer cell line) in a 5 mm NMR tube and placed in a 9.4 T NMR magnet. Signals of 1,4-¹³C-malate were detected by acquiring a set of ¹³C-MR spectra every 2 s with a 15 degree RF pulse. The amount of 1,4-¹³C-malate was calculated 30 s after mixing the hyperpolarized sodium 1,4-¹³C₂-fumarate into the medium containing cells. Further, the amount of soluble protein in the MDA-MB-231 cells was determined, and the amount of 1,4-¹³C-malate was normalized to the protein amount.

Using an identical protocol, a second study was performed in healthy breast cells (184B5, immortalized mammary gland cells). In this experiment, the amount was normalized to the amount of soluble protein in the 184B5 cells.

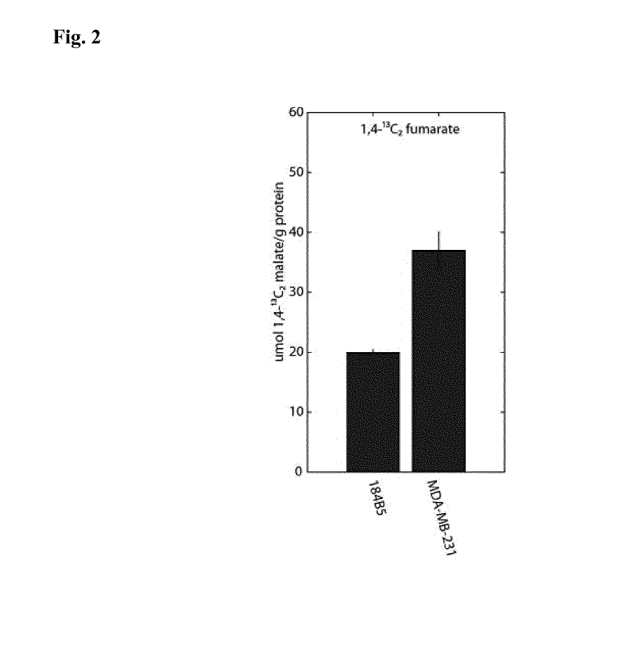

The results are compared in FIG. 2; the concentration of 1,4-¹³C-malate is significantly higher in MDA-MB-231 cells compared to healthy breast cells indicating an increased metabolism in the breast cancer cells.

### 3.10. Example 10

### Metabolism of Hyperpolarized 1,3-¹³C₂-ethyl Acetoacetate Measured in Intact Human Breast Cancer Cells, Human Prostate Cancer Cells and Human Lymphocytes

Substrates for carboxyl esterase are expected to be fast metabolizing compounds due to the high expression and activity of this class of enzymes. It was investigated whether a metabolic contrast could be found for the commercially available ¹³C-labelled substrate for the carboxyl esterase, 1,3-¹³C₂-ethyl acetoacetate, in cancer cells and lymphocytes.

### Materials and Methods

### Cell Growth and Harvest:

Human breast cancer cells (MCF-7) and human prostate cancer cells (PC-3) were grown in RPMI medium with 10% FBS and antibiotics. The cells were at confluence harvested by trypsination and washed once with 5 ml PBS. They were spun down and re-dissolved to a concentration of 20 million cells in 500 μl 40 mM phosphate buffer pH 7.3.

Human lymphocytes were purified from whole blood. Whole blood samples were drawn from a healthy volunteer into heparinised venous blood collection tubes (Venosafe vacumtubes from Terumo). For each experiment the blood was purified freshly: Three and a half 10 mL tubes of blood were pooled in each of four 50 mL Leucosep (Greiner) tubes prepared with 15 mL Histopaque 1077 (Sigma). The Leucosep tubes were spun for 15 min at 800× g at 24 C. The plasma was removed and the white blood cells were transferred to a 50 mL falcon tube by decanting and diluted 1:2 in PBS-Mg—Ca for a total volume of 50 mL. The cells were sedimented by centrifugation at 250×g for 60 min. Diluting with trypan blue and counting in hemocytometer determined the total number of lymphocytes before sedimentation. All cells were viable and the total number of cells in each cell batch was approx. 60 million. The lymphocytes were redissolved to a concentration of 20 million cells in 500 μl 40 mM phosphate buffer pH 7.3.

The cell suspensions were transferred to a 10 mm NMR tube and placed with connecting tubing in a 14.1 T magnet at 37 C.

### Hyperpolarized Marker:

Finland radical, carboxylic acid form (1 mg, 0.65 μmol) was dissolved in 1,3-¹³C₂ ethyl acetoacetate (50 μl, 51 mg, 0.30 mmol). To the solution was added a DMSO solution of the gadolinium complex ([alfa1,alfa4,alfa7-tris[(phenylmethoxy)methyl]-1,4,7,10-tetraazacyclododecane-1,4,7,10-tetraacetato(4-)]gadolinate(1-)]hydrogen) (0.8 mg of a 100 μmol/g solution). The concentration of radical and gadolinium were 13 mM and 1.6 mM respectively. 20 μmol of this composition was hyperpolarised under DNP conditions at 1.2 K in a 3.35 T magnetic field under irradiation with microwave (93.900 GHz). The polarization build-up constant was 750 s. The solid-state polarization was approx. 20%.

The hyperpolarized sample was dissolved in 5 ml phosphate buffer (40 mM, pH 7.3). The pH after dissolution was 7.3. Following dissolution, 1 ml of the substrate mixture was injected into 20 million cells in suspension. A series of 20 degree pulses every 2 s (56 scans in total) was acquired. The acquisition was started just before injection of the hyperpolarized substrate. Data are presented metabolic signal build-up as a function of time and as maximum metabolite signal.

### Results and Conclusions

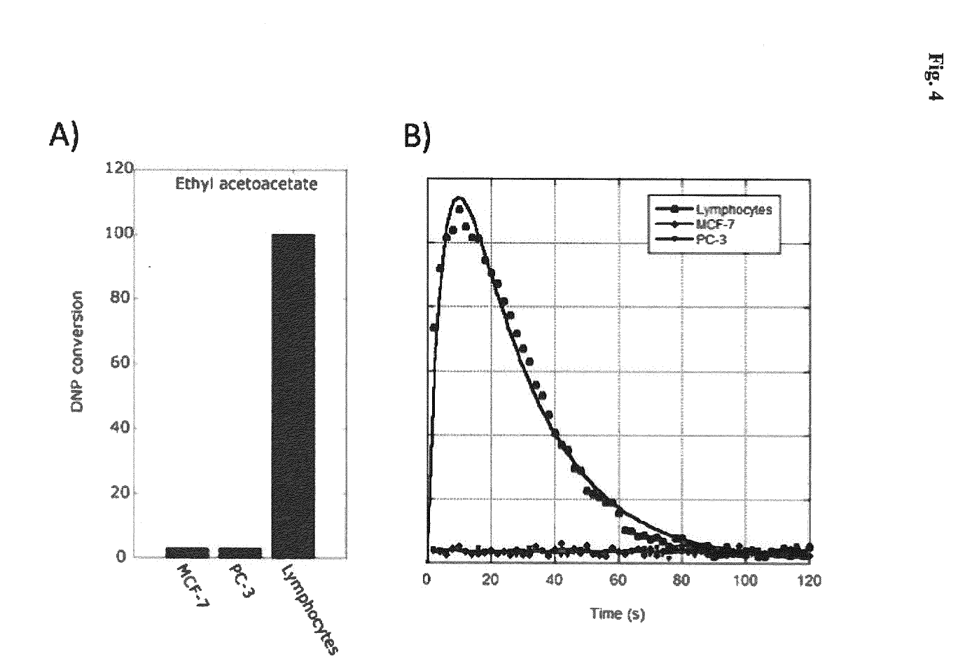

The numbers of produced hyperpolarized 1,3-¹³C₂-acetoacetate in lymphocytes, MCF-7 and PC-3 cells are shown in FIG. 4. It can be derived from this example that hyperpolarized 1,3-¹³C₂ ethyl acetoacetate is hydrolysed to 1,3-¹³C₂ acetoacetate in human lymphocytes but not in the two cancer cell types. Since no conversion can be detected in the cancer cells, the limit-of-detection (LoD) in the experiment is used for quantification of the metabolism of cancer cells in a background of lymphocytes. It can be appreciated that the metabolic conversion of hyperpolarized 1,3-¹³C₂ ethyl acetoacetate is approx. 33 times higher in the healthy lymphocytes than in cancer cells. This difference suggests that a large contrast between metastatic cancer and healthy tissue can be expected in a human lymph node using hyperpolarized 1,3-¹³C₂ ethyl acetoacetate as a metabolic contrast marker.

### 3.11. Example 11

### Metabolism of Hyperpolarized ¹³C₆-d₇-Glucose and 1-¹³C-Pyruvate in Intact Human Breast Cancer Cells, Human Prostate Cancer Cells and Human Lymphocytes

With a large metabolic contrast between a cancer metastasis and the dominating metabolising cell type in lymph nodes, lymphocytes, it is possible to discriminate between metabolising cells. A lymph node is, however, also made up of non-metabolising tissue such as connecting tissue. In order to being able to distinguish between non-metabolising tissue and cancer cells, a hyperpolarized marker is needed which will metabolise in all metabolically active cells but not in e.g. connecting tissue. The metabolism of human lymphocytes is similar to that of cancer metabolism in that these cells rely on aerobic glycolysis for energy (Macintyre and Rathmell, 2013, Cancer and Metabolism 1:5). Compounds such as glucose and pyruvate are thus expected to be metabolized particularly well in both cancer cells and lymphocytes. The real time metabolism of cancer cells was compared with that of the lymph node background metabolism. Two cancer cell types [breast cancer cells (MCF-7) and prostate cancer cells (PC-3)] and a mixture of B and T lymphocytes were used as model systems for the cell types present in a metastatic lymph node. The hyperpolarized substrates, ¹³C₆-d₇-glucose and 1-¹³C-pyruvate, were administered to the different cell types and compared to the results obtained with hyperpolarized 1,3-¹³C₂ ethyl acetoacetate of example 3.10.

### Materials and Methods

The cells were grown and harvested or purified as described in example 3.10. The experiments were performed with single substrates administered to 20 million MCF-7 cells, PC-3 cells or lymphocytes dissolved in a volume of 500 μl 40 mM phosphate buffer pH 7.3. The cell suspensions were transferred to a 10 mm NMR tube and placed with connecting tubing in a 14.1 T magnet at 37° C.

The Hyperpolarized ¹³C₆-d₇-Glucose was Prepared as Follows:

¹³C₆-d₇-glucose (22.8 mg, 0.118 mmol) was dissolved in polarization medium (25.0 mg). The polarization medium was made of Ox063 radical (19.1 mg, 13.3 μmol) was dissolved in 465 μl water. To this solution was added Gadoteridol (40 mg of 50 μmol/g solution in water). Concentrations: [Ox063]=26.5 mM, [Gd]=4 mM. The total weight of the glucose preparation was: 47.8 mg yielding 2.47 mmol glucose/g preparation.

### The Hyperpolarized 1-¹³C-Pyruvate was Prepared as Follows:

1-¹³C pyruvic acid (55 μl, 70.0 mg) was mixed with Finland radical, sodium salt (1.5 mg, 0.94 μmol) and Gadoteridol (2.1 mg of 50 μmol/g solution in water). Concentrations of radical and Gadolinium were: 17 mM; 2 mM.

20 μmol of either substrate was polarized in a 3.3 T polarizer operating at 93.905 MHz and approx. 1.2 K. Following 45 minutes polarization the samples were dissolved in 5 ml 40 mM phosphate buffer pH 7.3. In the experiments with pyruvic acid an equimolar addition of NaOH was added to neutralize the acid. 2 ml of the dissolved substrate mixture was injected into the cells in suspension. A series of 20 degree pulses every 2 s (56 scans in total) was acquired. The acquisition was started just before injection of the hyperpolarized substrate. Data are presented as maximum metabolite signals, hyperpolarized 1-¹³C lactate produced from either glucose or pyruvate.

### Results and Conclusions

The numbers of the produced hyperpolarized products, 1-¹³C lactate from either ¹³C₆-d₇-glucose or 1-¹³C-pyruvate in the two human cancer cell types and in human lymphocytes are given in Table 1.

TABLE 1
Metabolic conversion of hyperpolarised ¹³C₆-d₇-glucose and
1-¹³C-pyruvate measured in 20 million of the different cell types
as indicated. The metabolic product is quantified in a.u.
Metabolic conversion (a.u.)

| Substrate | MCF-7 cells | PC-3 cells | Lymphocytes |
|---|---|---|---|
| 1-¹³C-pyruvate | 1000 | 200 | 40 |
| ¹³C₆-d₇-glucose | 500 | 200 | 10 |

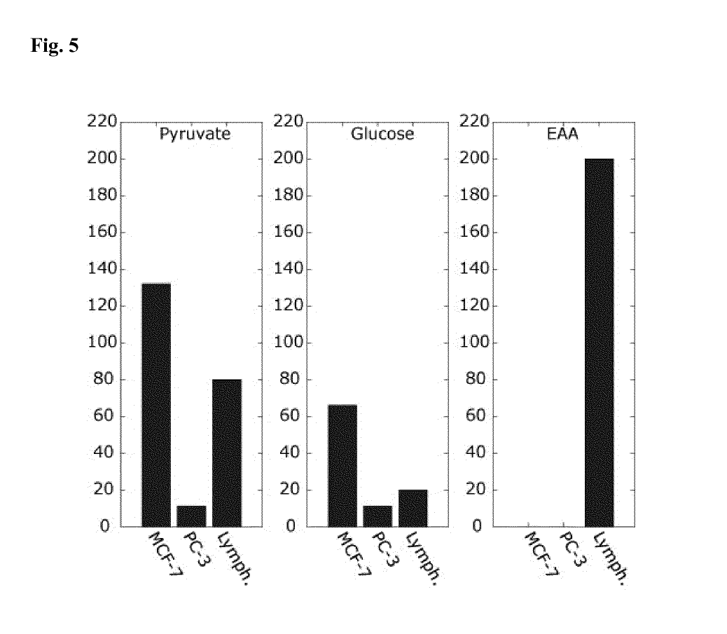

The conversion of both hyperpolarized 1-¹³C-pyruvate and hyperpolarized ¹³C₆-d₇-glucose is, when quantified by cell number, higher in both cancer cell types compared to lymphocytes. Especially the conversion in breast cancer cells is 50 times higher of both substrates than in lymphocytes. However, lymphocytes are much smaller than both cancer cells. A contrast between a metastatic breast or prostate cancer in surrounding lymphocytes is therefore better predicted considering the different cell sizes (MCF-7 cells are ˜18 μm in diameter, PC-3 cells are ˜24 μm in diameter and lymphocytes are on an average ˜7.2 μm in diameter) (Arya et al. (2012) Lab Chip, 12, 2362-2368; Kolios and G. Czarnota, (2008), physics publications and Research paper 11; Abbas A K and Lichtman A H (2003), Cellular and Molecular Immunology (5th ed.). Saunders, Philadelphia). The considerable size difference between the cancer cells and the surrounding lymphocytes leads to significant differences in the number of cells packed inside an otherwise identical volume of tissue. It follows from the cell diameters that 1 μl tissue contains 330.000 MCF-7 cells, 140.000 PC-3 cells or 5 million lymphocytes. Using conventional MRI techniques, an image of a tissue can be made with spatial resolution (S. Josan et al., (2012) NMR in biomedicine, vol 25(8): 993-999). A standard experiment was assumed, which has a spatial resolution of 2×2×2 mm. This corresponds to a voxel volume of 8 μl or converted to cell numbers: 2.6 million MCF-7 cells, 1.1 million PC-3 cells or 40 million lymphocytes. The actual contrast obtained between MCF-7 or PC-3 cells surrounded by lymphocytes in a spatially resolved lymph node using either hyperpolarized ¹³C₆-d₇-glucose, 1-¹³C-pyruvate or 1,3-¹³C₂ ethyl acetoacetate (values for 20 million cells obtained from example 1) is depicted in FIG. 5.

With the combination of a hyperpolarized marker providing information on metabolic activity and a hyperpolarized marker, which shows a contrast between a cancer cell and a lymphocyte, it is possible to diagnose a breast or prostate metastasis in the surroundings of metabolizing lymph node cells, lymphocytes. In particular, the combination of the metabolic marker hyperpolarized 1-¹³C-pyruvate, which is taken up and converted fast to hyperpolarized 1-¹³C-lactate in both cancer cells and lymphocytes, with the Carboxyl esterase substrate hyperpolarized 1,3-¹³C₂ ethyl acetoacetate, which is hydrolysed to 1,3-¹³C₂ acetoacetate in lymphocytes only, is expected to allow a breast or prostate metastasis to be detected in lymph nodes.

### 3.12. Example 12

### Use of the Combination of Hyperpolarized 1-¹³C-Pyruvate and 1,3-¹³C₂-Ethyl Acetoacetate to Distinguish Human Breast Cancer Cells from Human Lymphocytes

The real time metabolism of breast cancer cells was compared with the metabolism of the lymph node background in model systems containing either only cancer cells (100% cancer), only lymphocytes (0% cancer) or a combination of the two cell types (50% cancer) in cellular concentrations expected to be present in a spatially resolved lymph node containing a macro metastasis. In the clinic, the interest is focused on macro metastasis since these are statistically those, which indicate aggressive cancers (P. Blumencranz, Surg Oncol Clin N Am 20 (2011) 467-485).

### Materials and Methods

The model system was designed assuming a macro metastasis, which is defined to have a minimum diameter of 2 mm (P J. Diest et al., (2010) Breast disease 31: 65-81). In a 2×2×2 mm voxel, 50% of the volume will be cancer cells assuming that the macro metastasis is shaped as a sphere. Three experimental systems were established and carried out: 1) 2 million MCF-7 cells (corresponding to 100% cancer cells in a 2×2×2 voxel), 2) 1 million MCF-7 cells and 20 million lymphocytes (corresponding to 50% cancer cells in a 2×2×2 voxel) and 3) 40 million lymphocytes (corresponding to 0% cancer cells in a 2×2×2 voxel). The cells were grown and harvested or purified as described in example 3.10. The DNP preparations of 1-¹³C-pyruvate and 1,3-¹³C₂-ethyl acetoacetate were made as described in examples 3.10. and 3.11. The substrates (20 μmol each) were co-polarized. The polarization, dissolution and administration to the cells were made as in example 3.11.

### Results and Conclusions

The numbers of the produced hyperpolarized products, 1-¹³C lactate from 1-¹³C-pyruvate and 1,3-¹³C₂ acetoacetate from 1,3-¹³C₂ ethyl acetoacetate in the three experiments are given in Table 2.

TABLE 2
Metabolic conversion measured with hyperpolarized substrates
in the three model voxels containing 0%, 50% or 100% breast
cancer cells (MCF-7) in the background of human lymphocytes.
The metabolic product is quantified in a.u.
Metabolic conversion (a.u)

| | 100% cancer | 50% cancer | 0% cancer |
|---|---|---|---|
| Substrate | cells | cells | cells |
| 1-¹³C-pyruvate | 337 | 1853 | 2620 |
| 1,3-¹³C₂ ethyl acetoacetate | 14* | 632 | 1027 |

*No metabolic conversion could be detected for 1,3-¹³C₂ ethyl acetoacetate in the experiment with 100% cancer cells and the limit of detection (LoD) of the experiment is therefore used for quantification purposes.

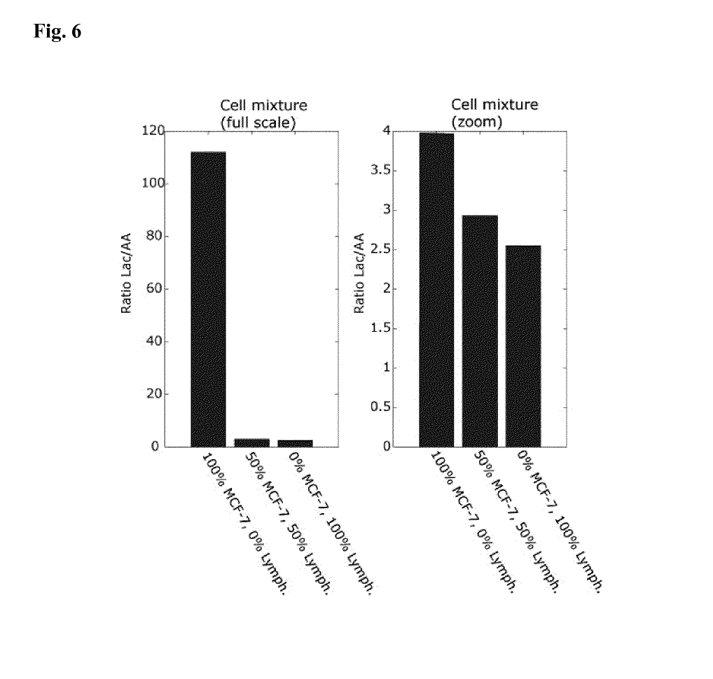

A significant contrast of 24 times can be obtained in a metabolic ratio map of the detected hyperpolarized metabolic products in the experiment corresponding to a 2×2×2 mm voxel containing 100% metastatic breast cancer relative to that of 0% metastatic breast cancer in surrounding lymphocytes, FIG. 6. A contrast of 20% (compared to 30% theoretically possible) is measured for the experiment corresponding to 50% metastatic breast cancer cells in the background of lymphocytes. The voxel size may be chosen to optimize the diagnosis to match the precise size of a metastasis of interest and thereby increase the contrast towards that obtained for 100% cancer.

### 3.13. Example 13

### Comparison of Short Chain Esters as Substrates for Carboxyl Esterase CE-1 and CE-2

Different classes of short chain esters, fulfilling the requirements for the hyperpolarization technique (stable, soluble, low molecular weight compounds (<400 Da), were evaluated as substrates for carboxyl esterases with the aim to identify esters as similar or better substrates for carboxyl esterases than 1,3-¹³C₂ ethyl acetoacetate. In most human cell types, including a wide range of cancer cells and lymphocytes, the activity of carboxyl esterase is dominated by the activities of isoforms 1 and 2 (CE-1 and CE-2). To measure exclusively the hydrolysis of carboxyl esterase from the CE-1 isoform, an isolated enzyme from porcine liver was applied. To measure almost exclusively the contribution of carboxyl esterase activity from the CE-2 isoform, human breast cancer cells (MCF-7) were employed. These cells are known from literature to express CE-2 but not to express CE-1 (Byun et al, 2008, Cancer letters 266:238-48). A group of esters was investigated as substrates for CE-1, which contained different structural features. These consisted of esters of un-substituted acids (examples are ethyl acetate, ethyl butyrate, methyl butyrate), esters of acids substituted on C2 (examples are ethyl lactate and ethyl 2-acetoxy propanoate), esters of acids substituted on C3 (examples are ethyl 3-hydroxy butyrate and ethyl 3-acetoxy butanoate), esters of ring closed acids (example ethyl pyroglutamate), esters of di-acids (examples are diethyl succinate and dimethyl succinate), esters of tri-acids (example triethyl citrate), ethyl esters of beta-keto acids (example ethyl acetoacetate). Also esters with large alcohol groups and small acid groups were investigated. In this group, the acid part of the molecule was always acetate and different structural properties of the alcohol were investigated. These consisted of esters of un-substituted alcohols (examples ethyl acetate and butyl acetate), esters of branched chain alcohols (examples t-butyl acetate, ethyl 3-acetoxy butanoate and 2-acetoxy propionate), esters of aromatic alcohols (example benzyl acetate), esters of diols (example ethyleneglycol diacetate), esters of triols (example triacetin). Esters of alpha-keto acids were too unstable to be investigated in this assay. Esters of larger alcohols are not water soluble and therefore not relevant substrates for the purpose of the invention. Similarly, a group of esters which contained different structural features was investigated as substrates for CE-2. CE-2 substrates are described in the literature to consist of a small acid group and a larger alcohol group. All included esters were acetate esters (i.e. esters of acetic acid) in the interest of keeping the acid moiety the smallest possible. The acetate esters measured for CE-2 activity were therefore the same as those measured in the second group of possible substrates for CE-1.

### Materials and Methods

The CE-1 experiments were performed with commercially available CE-1 from porcine liver. Individual samples of the substrates were all prepared the following way: 26 μmol was weighted in an eppendorf tube. 550 μl deuterated phosphate buffer (200 mM, pH 7.5) was added and the substrate was brought into solution by whirl mixing. The solution was transferred to a 5 mm NMR tube and inserted in the spectrometer at 37° C. A reference experiment was acquired on the substrate solution without enzyme after which 10 μl of an esterase stock solution (37 U/ml in deuterated phosphate buffer) was added in the top of the tube. The sample was mixed and returned to the spectrometer. A series of thermal 1D ¹H NMR experiments were recorded every 5 min for 60 min. The data were analysed with MNOVA software and measured as percentage conversion of substrate calculated to account for the amount of substrate converted over 60 min. The numbers are given in μmol/min.

The CE-2 experiments were performed with lysed breast cancer cells (MCF-7). MCF-7 cells were grown in RPMI medium with 10% FBS and antibiotics. They were at confluence harvested by trypsination and washed once with 5 ml PBS. They were spun down and resuspended in PBS to a concentration of 20 mill. cells/ml. The cells were hereafter sonicated on ice (50% amplitude, 1 min, 3 sec pulse). 250 μl sonicated cell suspension was mixed with 250 μl 400 mM phosphate buffer pH 7.3. This solution was transferred to a 5 mm NMR tube and warmed on a water bath to 37° C. The substrate was added (50 μl, 60 mM) and the tube turned for mixing before it was inserted into the spectrometer. An initial spectrum was acquired and the sample was hereafter placed at 37° C. for 1 hour in a water bath. The sample was again inserted into the magnet and a second spectrum was recorded. The data was analysed with MNOVA software and measured as percentage conversion of substrate calculated to account for the amount of substrate converted over 60 min. The numbers are given in μmol/hour.

### Results and Conclusions

### CE-1 Substrates:

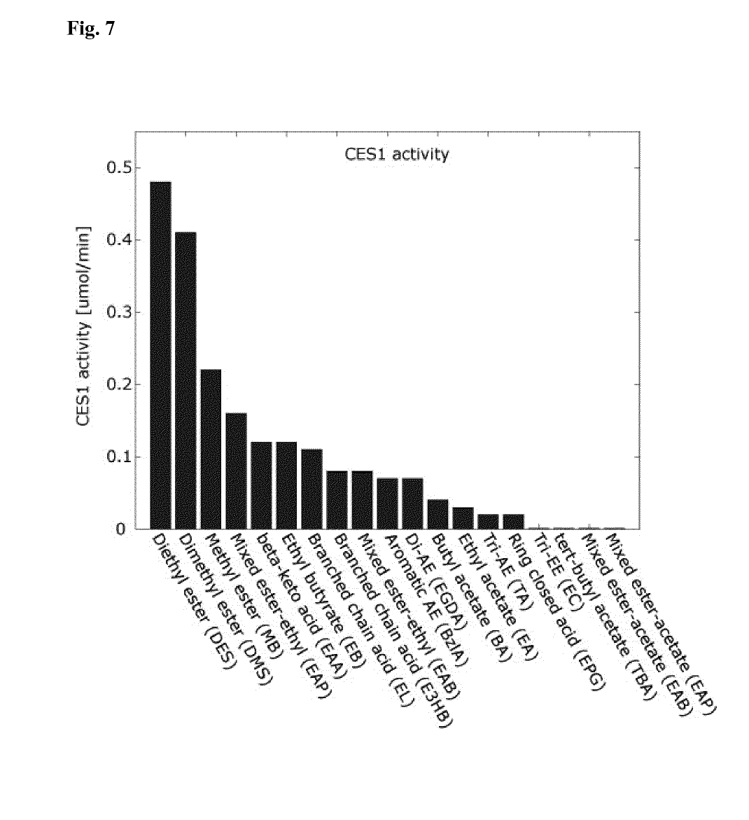

Almost all of the investigated esters were substrates for CE-1, however with very different turnover rates, see Table 3 and FIG. 7. The overall fastest converted substrates were dimethyl and diethyl succinate, which was more than a factor of 2 faster converted than the next class of tested preferred substrates, methyl butyrate and ethyl lactate acetate and more than ten times faster converted than ethyl acetate. Conversion of the ethyl ester of pyroglutamate was barely detectable and no hydrolysis could be detected for triethyl citrate.

TABLE 3
CE-1 activities of a variety of short chain esters measured
with isolated CE-1 enzyme from porcine liver.

| | k | | k | | k |
|---|---|---|---|---|---|
| | (μmol/ | | (μmol/ | | (μmol/ |
| Substrate | min) | Substrate | min) | Substrate | min) |
| Ethylacetate | 0.03 | Dimethyl succinate | 0.41 | Butyl acetate | 0.04 |
| (EA) | | (DMS) | | (BA) | |
| Ethylbutyrate | 0.12 | Diethyl succinate | 0.48 | Tri-acetin (TA) | 0.02 |
| (EB) | | (DES) | | | |
| Methylbutyrate | 0.22 | Triethyl citrate (EC) | 0 | Tert-butyl acetate | 0 |
| (MB) | | | | (TBA) | |
| Ethyl | 0.12 | ethyl 3-acetoxy | 0.08 | Benzyl acetate | 0.07 |
| acetoacetate | | butanoate (ethyl ester) | | (BzlA) | |
| (EAA) | | (EAB) | | | |
| Ethyl lactate | 0.11 | ethyl 2-acetoxy | 0.16 | ethyl 3-acetoxy | 0 |
| (EL) | | propanoate (ethyl | | butanoate (acetate | |
| | | ester) (EAP) | | ester) (EAB) | |
| Ethyl 3- | 0.08 | | | ethyl 2-acetoxy | 0 |
| Hydroxybutyrate | | | | propanoate | |
| (E3HB) | | | | (acetate ester) | |
| | | | | (EAP) | |
| Ethyl | 0.02 | | | Ethyleneglycol | 0.07 |
| pyroglutamate | | | | diacetate (EGD) | |
| (EPG) | | | | | |

Symmetric diethyl or dimethyl esters represented by methyl and ethyl succinate are the best (of the investigated) substrates for CE-1. Low molecular weight monoesters are in general good substrates including esters of branched chain carboxylic acids. Ethyl acetate that has a very short chain on both the alcohol and acid side of the ester is not a good substrate for CE-1. Triethyl citrate and the ring closed ethyl pyroglutamate are only hydrolyzed to a very low extent.

### CE-2 Substrates:

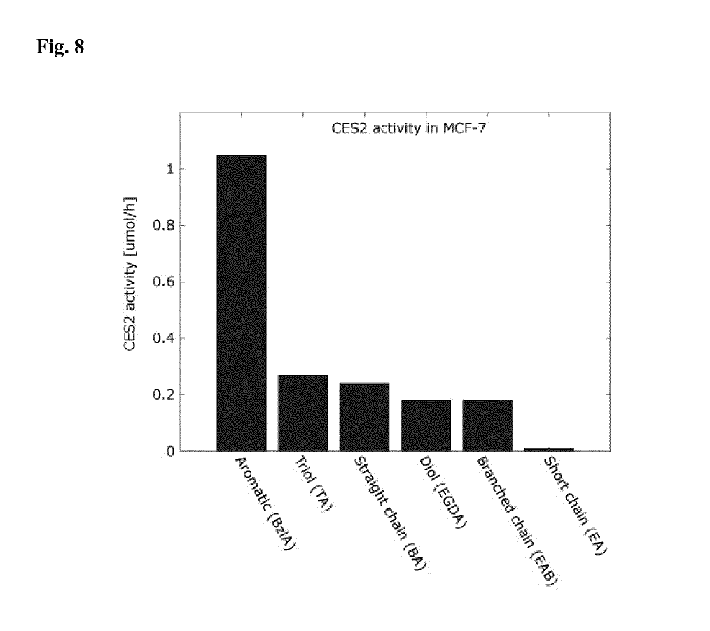

Almost all of the investigated esters were substrates for CE-2, however with different turnover rates, see Table 4 and FIG. 8. Of seven investigated compounds, one was hydrolysed particularly fast (Benzyl acetate), four compounds were hydrolysed to a similar extent (butyl acetate, Tri-acetin and the acetate esters of ethyl 2-acetoxy propanoate and ethyl 3-acetoxy butanoate) while ethyl acetate was not hydrolysed. That ethyl acetate was not a substrate for CE-2 is in agreement with this substrate having a small alcohol group (ethanol), which is not preferred for the CE-2 isoform of carboxyl esterase (Imai et al., 2006, Drug Metab. Pharmacokinet 21(3): 173-85).

TABLE 4
CE-2 activities of acetate esters measured
in lysed human breast cancer cells (MCF-7).

| Substrate | k(μmol/min) | Substrate | k(μmol/min) |
|---|---|---|---|
| Ethylacetate (EA) | 0 | Benzylacetate | 1.05 |
| | | (BzlA) | |
| Tri-acetin (TA) | 0.27 | Ethylenglycol | 0.18 |
| | | diacetyl (EGDA) | |
| Butylacetate | 0.24 | ethyl 3-acetoxy | 0.18 |
| (BA) | | butanoate | |
| | | (acetate ester) | |
| | | (EAB) | |

Aromatic acetate esters, represented by benzyl acetate, are the best (of the investigated) substrates for CE-2.

### 3.14. Example 14

### Differences in Carboxyl Esterase Activities Measured in Human Breast Cancer Cells, Human Prostate Cancer Cells and Human Lymphocytes

The activity of carboxyl esterase was measured in human cancer cells (breast and prostate) and compared with that of the majority cell type in lymph nodes, human B and T lymphocytes. The purpose of the comparison was to find metabolic differences between cancer cells and normal cells present in a metastatic lymph node.

### Materials and Methods

The hydrolysis of the substrates was investigated in cell extracts. To test substrates without isotope labelling for carboxyl esterase activity in a cellular background, a ¹H NMR assay was applied. Human breast cancer cells (MCF-7) and human prostate cancer cells (PC-3) were grown in RPMI medium with 10% FBS and antibiotics. They were at confluence harvested by trypsination and washed once with 5 ml PBS. They were spun down and resuspended in PBS to a concentration of 20 mill. cells/ml. The cells were hereafter sonicated on ice (50% amplitude, 1 min, 3 sec pulse). Human lymphocytes were purified from whole blood. Whole blood samples were drawn from healthy volunteer into heparinised venous blood collection tubes (Venosafe vacumtubes from Terumo). For each experiment, the blood was purified freshly: Three and a half 10 mL tubes of blood were pooled in each of four 50 mL Leucosep (Greiner) tubes prepared with 15 mL Histopaque 1077 (Sigma). The Leucosep tubes were spun for 15 min at 800× g at 24 C. The plasma was removed and the white blood cells were transferred to a 50 mL falcon tube by decanting and diluted 1:2 in PBS-Mg—Ca for a total volume of 50 mL. The cells were sedimented by centrifugation at 250×g for 60 min. Diluting with trypan blue and counting in hemocytometer determined the total number of lymphocytes before sedimentation. All cells were viable and the total number of cells in each cell batch was approx. 60 million. The lymphocytes were redissolved to a concentration of 20 million cells/ml. The cells were hereafter sonicated on ice (50% amplitude, 1 min, 3 sec pulse).

250 μl sonicated cell suspension was mixed with 250 μl 400 mM phosphate buffer pH 7.3. This solution was transferred to a 5 mm NMR tube and warmed on a water bath to 37° C. The substrate was added (50 μl, 60 mM) and the tube turned for mixing before it was inserted into the spectrometer. The data was analyzed with MNOVA software and measured as percentage conversion of substrate calculated to account for the amount of substrate converted over 60 min; numbers are given in μmol/hour.

### Results

A summary of measured esterase activities in the three different cell types is given in Tables 5 and 6.

TABLE 5
Metabolic conversion of esters in 5 million lysed breast
cancer cells (MCF-7), lysed prostate cancer cells (PC-3)
and lysed lymphocytes. Numbers are given in umol/hour.

| | Cell extract | | |
|---|---|---|---|
| | Breast cancer | Prostate cancer | Lymphocytes |
| Substrate | cells MCF-7 | cells PC-3 | (B + T cells) |
| Diethyl succinate | 0.12 | 0.48 | 0.60 |
| Methyl butyrate | 0.42 | 0.51 | 0.90 |
| Ethyl acetoacetate | 0* | 0* | 0.15 |
| Tri-acetin | 0.27 | 1.8 | 0.18 |
| Benzyl acetate | 1.05 | 22 | 0.39 |

*No conversion was measured under the given experimental conditions.

TABLE 6
Metabolic conversion of ester with double functionality
(acetate and ethyl ester) in 5 million lysed breast cancer
cells (MCF-7), lysed prostate cancer cells (PC-3) and
lysed lymphocytes. Numbers are given in μmol/hour.

| | Cell extract | | |
|---|---|---|---|
| | Breast cancer | Prostate cancer | Lymphocytes |
| | cells MCF-7 | cells PC-3 | (B + T cells) |
| | Ratio | Ratio | Ratio |
| Substrate | acetate/ethyl | acetate/ethyl | acetate/ethyl |
| Ethyl 3-HB acetate | 2 | 9 | 1 |

In breast cancer cells relative to lymphocytes measured on a cell number basis, ethyl acetatoacetate (EAA) gives the highest contrast. The much better CE-1 substrate, diethyl succinate also provides a negative contrast (the conversion is lower in breast cancer cells than in lymphocytes), which is approx. 3 times lower than that of EAA. Three substrates provide a positive contrast (the conversion is higher in breast cancer cells than in lymphocytes). Of the positive contrast esters, the aromatic benzyl acetate is the best.

In prostate cancer cells relative to lymphocytes measured on a cell number basis, ethyl acetatoacetate (EAA) gives the highest negative contrast. Three substrates give a high positive contrast, of which the aromatic benzyl acetate is much preferred with an outstanding contrast of almost 60 times higher metabolic conversion in prostate cancer cells compared to lymphocytes.

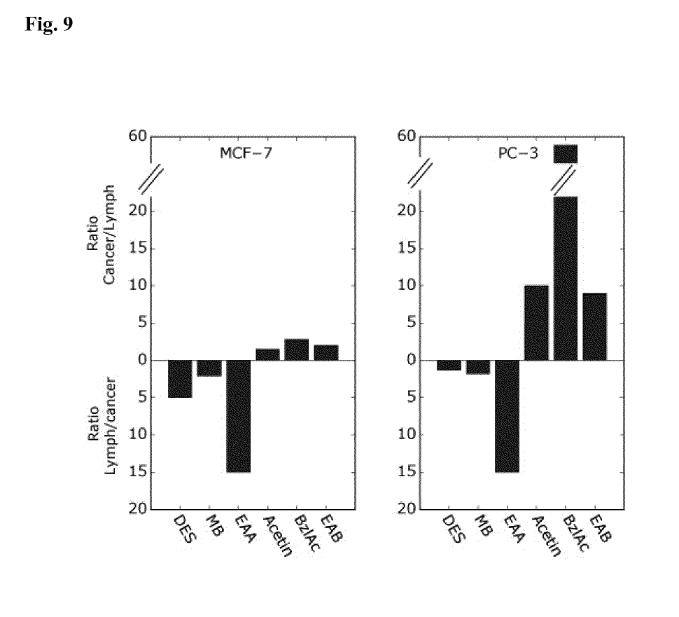

All ethyl esters give a negative contrast when the cell number is equal as shown in FIG. 9. When considering that lymphocytes are smaller than cancer cells, this contrast will increase in a tissue sample of a given size since the number of lymphocytes will be larger than the number of cancer cells. Even though the contrast is highest with ethylacetoacetate, it is preferred to use diethylsuccinate since the overall activity of CE-1 towards this substrate is higher, which provides a higher signal to noise.

1. A method of diagnosing a cancer metastasis in a lymph node tissue sample, wherein said method is carried out on an excised lymph node tissue sample obtained from a patient suffering from cancer and wherein said method comprises the following steps:
a) Contacting said tissue sample with a combination of (i) a hyperpolarized metabolic marker indicating metabolically active cells and (ii) a hyperpolarized metabolic marker allowing a distinction between lymphocytes and cancer cells;
b) Obtaining an NMR spectrum and/or an MR image of the metabolic products of (i) and (ii) comprised in said tissue sample;
c) Determining the ratio rS of the metabolic products of (i) to the metabolic products of (ii);
d) Comparing the ratio rS obtained in step c) to a reference ratio rR of the metabolic products of (i) to the metabolic products of (ii) obtained in a lymph node tissue sample consisting of healthy cells; and
e) Assigning cancer to said tissue sample based on the comparison carried out in step d), wherein a difference in the ratio rS to the ratio rR indicates the presence of a cancer metastasis in said tissue sample.

2. The method according to claim 1, wherein (ii) is a carboxylate ester of a molecular weight of ≦400 Da.

3. A method of diagnosing a cancer metastasis in a lymph node tissue sample, wherein said method is carried out on an excised lymph node tissue sample obtained from a patient suffering from cancer and wherein said method comprises the following steps:
a) Contacting said tissue sample with a combination of (i) a hyperpolarized metabolic marker indicating metabolically active cells and (ii) a hyperpolarized metabolic marker exhibiting either a higher conversion in cancer cells compared to lymphocytes or a lower conversion in cancer cells compared to lymphocytes;
b) Obtaining an NMR spectrum and/or an MR image of the metabolic products of (i) and (ii) comprised in said tissue sample;
c) Determining the ratio rS of the metabolic products of (i) to the metabolic products of (ii);
d) Comparing the ratio rS obtained in step c) to a reference ratio rR of the metabolic products of (i) and (ii) obtained in a lymph node tissue sample consisting of healthy cells; and
e) Assigning cancer to said tissue sample based on the comparison carried out in step d), wherein a ratio of rS:rR of >1 indicates the presence of a cancer metastasis in said tissue sample if (ii) exhibits a higher conversion in cancer cells compared to lymphocytes or wherein a ratio of rR:rS of >1 indicates the presence of a cancer metastasis in said tissue sample if (ii) exhibits a lower conversion in cancer cells compared to lymphocytes.

4. The method according to claim 1, wherein said cancer is selected from the group consisting of breast cancer, prostate cancer, head and neck cancer and colon cancer.

5. The method according to claim 3, wherein said cancer is breast cancer.

6. The method according to claim 3, wherein said cancer is prostate cancer.

7-8. (canceled)

9. The method according to claim 3, wherein the (ii) hyperpolarized metabolic marker exhibiting a higher conversion in cancer cells compared to lymphocytes is an ester selected from esters of acetic acid with a molecular weight of ≦400 Da, wherein the alcohol part comprises a straight or branched alkyl chain and/or aromatic group.

10. (canceled)

11. The method according to claim 3, wherein the (ii) hyperpolarized metabolic marker exhibiting a higher conversion in cancer cells compared to lymphocytes is acetin, benzyl acetate, or ethyl 3-acetoxybutanoate.

12. The method according to claim 3, wherein the (ii) hyperpolarized metabolic marker exhibiting a slower conversion in cancer cells compared to lymphocytes is an ester selected from ethyl or methyl esters or a carboxylic acid with a molecular weight of ≦400 Da, wherein the acid part comprises a straight or branched alkyl chain and/or aromatic group.

13. (canceled)

14. The method according to claim 3, wherein the (ii) hyperpolarized metabolic marker exhibiting a slower conversion in cancer cells compared to lymphocytes is diethyl succinate, methyl butyrate or ethyl acetoacetate.

15-16. (canceled)

17. The method according to claim 1, wherein (i) is glucose, pyruvate, lactate, fumarate, malate, an alpha-keto-acid, or an alpha amino acid.

18. The method according to claim 1, wherein (i) is glucose or pyruvate.

19-20. (canceled)

21. The method according to claim 3, wherein said method is carried out while said patient, from which said tissue sample has been obtained, is undergoing surgery for a primary tumor.

22-24. (canceled)

25. A kit comprising (i) a metabolic marker indicating metabolically active cells and (ii) a metabolic marker allowing a distinction between lymphocytes and cancer cells.

26-27. (canceled)

28. The kit according to claim 25, wherein (ii) is acetin, benzyl acetate, ethyl 3-acetoxybutanoate, diethyl succinate, methyl butyrate, or ethyl acetoacetate.

29. (canceled)

30. The kit according to claim 25, wherein (i) is glucose, pyruvate, lactate, fumarate, malate, an alpha-keto-acid, or an alpha amino acid.

31-35. (canceled)

36. The method according to claim 1, wherein said method is carried out while said patient, from which said tissue sample has been obtained, is undergoing surgery for a primary tumor.

37. The method according to claim 3, wherein (i) is glucose, pyruvate, lactate, fumarate, malate, an alpha-keto-acid, or an alpha amino acid.

38. The method according to claim 3, wherein (i) is glucose or pyruvate.
